(19) United States
(12) Patent Application Publication
(10) Pub. No.: US 2009/0214533 A1
(43) Pub. Date: Aug. 27, 2009

(54) METHODS FOR CONVERTING OR INDUCING PROTECTIVE IMMUNITY

(75) Inventor: Raphael Clynes, West Nyack, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/372,106

(22) Filed: Feb. 17, 2009

Related U.S. Application Data
(63) Continuation of application No. PCT/US2007/018129, filed on Aug. 15, 2007.
(60) Provisional application No. 60/838,608, filed on Aug. 17, 2006.
(60) Provisional application No. 60/847,591, filed on Sep. 27, 2006.

(51) Int. Cl. A61K 39/395; A61P 35/00

(52) U.S. Cl. 424/133.1; 424/173.1

(57) ABSTRACT

The invention is based in part on the finding that suppressing regulatory T cell function is needed in order to convert passive immunity into active antigen-specific immunity. Generally, the methods of the invention comprise at least the combination of: (1) increasing the amount of immune complexes in the subject, wherein the immune complex comprises a target antigen and a immunoglobulin molecule comprising (i) a variable region specific to the target antigen and (ii) a Fc receptor binding region; and (2) inhibiting regulatory T cell function or decreasing/depleting the regulatory T cell population in the subject.

This application is a continuation of International Patent Application No. PCT/US2007/018129, filed Aug. 15, 2007, which claims the benefit of U.S. Provisional Patent Application No. 60/838,608, filed Aug. 17, 2006, and U.S. Provisional Patent Application No. 60/847,591, filed Sep. 27, 2006. All patents, patent applications, and publications cited herein are hereby incorporated by reference in their entirety into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

The immune system responds to harmful pathogens while remaining tolerant to autologous tissues. Thus, organisms have a biological defense system to remove exogenous and harmful materials and simultaneously have established self-tolerance. Immune responses are activated and regulated by interactions between T-lymphocytes, B-lymphocytes, antigen-presenting cells (APCs), and antibodies.

An APC, such as a macrophage or dendritic cell, is an important link in the induction of active immunity. The APC breaks down endocytosed antigens and subsequently presents the antigen peptides on its surface via a major histocompatibility complex (MHC) molecule. Helper T cells with the appropriate T cell receptor (TCR) recognize exogenous antigens bound to MHCs, resulting in the T cell binding to the APC, activation of the T cell, and subsequent secretion of cytokines. These cytokines then activate and differentiate B-cells in order for the latter to become antibody-producing cells wherein the antibodies later mark foreign materials for destruction by other immune cells. Cytokines secreted by Helper T cells (T_{H} cells) also accelerate the differentiation of cytotoxic T cells (T_{C} cells; also called killer T cells). In a subject's body, cytotoxic T cells aid in removing cells that have been infected by viruses or have been transformed by cancer for the cells have not yet adapted to evade the immune system. Thus, T_{H} cells play a central role and recognize antigens to be targeted, helping the body to acquire specific immunity to the antigens.

Regulatory T cells (Tregs; also known as suppressor T cells) are a specialized subpopulation of T cells that act to suppress activation of the immune system and thereby maintain immune system homeostasis and tolerance to self Interest in regulatory T cells has been heightened by evidence from experimental mouse models demonstrating that the immunosuppressive potential of these cells can be harnessed therapeutically to treat autoimmune diseases and facilitate transplantation tolerance or specifically eliminated to potentiate cancer immunotherapy.

### SUMMARY OF THE INVENTION

The invention is based in part on the finding that suppressing regulatory T cell function (or depleting/decreasing T regulatory cell number in a subject) is needed in order to convert passive immunity provided by antibody administration into an active immunity (as manifested for example by specific T helper and T cytotoxic responses). In certain aspects, the methods of the invention comprise at least the combination of: (1) increasing the amount of immune complexes in the subject, wherein the immune complex comprises a target antigen and a immunoglobulin molecule comprising (i) a variable region specific to the target antigen and (ii) a Fc receptor binding region; and (2) inhibiting regulatory T cell function or decreasing/depleting the regulatory T cell population in the subject. In one aspect, the conversion from passive immunity to active immunity is manifested by the induction or enhancement of T cell specific immune responses to the antigen target of the antibodies.

Thus, the methods of the invention can comprise co-administration of anti-tumor/anti-pathogen/anti-disease marker antibodies with substances (“Treg agents”″ that inhibit T regulatory cell function or deplete or diminish T regulatory cell populations in order to treat cancer, disease, or pathogenic infections. Without being bound by theory, one mechanism of action may include engagement and activation of FcR bearing cellular effectors (including, for example, macrophages, neutrophils, and NK cells) via the administration of the anti-tumor/anti-pathogen antibodies that form immune complexes with target antigens, such that ADCC (antibody-dependent cellular cytotoxicity) responses against the tumor or pathogen occurs. Additionally or alternatively, another mechanism of action may comprise antibodies activating components of the complement system leading to lysis of the tumor cell (called complement dependent cytotoxicity or CDC). Anti-tumor antibodies and antibodies directed at pathogenic antigens may also induce protective immunity through activation of Fc receptors on dendritic cells or other APCs, thus promoting T cell responses. For example, anti-tumor antibodies can bind tumor antigens in vivo. This can result in the formation of soluble tumor antigen-containing immune complexes, antibody opsonized tumor cells, and tumor cell fragments. However, administration of therapeutic antibodies alone is not sufficient to induce active immunity against the target of the antibodies, rather, the invention has determined that a conversion to active immunity requires the co-administration of therapeutic antibodies with an agent that can temporarily inhibit T regulatory cell function or temporarily deplete/diminish the T regulatory cell population.

In one aspect, the invention provides a method for converting a passive immunization against a target antigen into active immunity against the target antigen in a subject, the method comprising: (a) administering an effective amount of an agent, wherein the agent decreases the activity or function of a regulatory T cell or substantially depletes the regulatory T cell population in the subject, and (b) increasing immune complex formation or immune complex number in the subject, wherein the immune complex comprises (i) an antibody or antibody fragment that comprises at least a portion of an immunoglobulin variable region that specifically binds to the target antigen and at least a portion of immunoglobulin constant region that can bind to an Fc-receptor; thereby inducing, activating, or stimulating T helper and or T cytotoxic cells that have T cell receptors specific to the target antigen in the subject. The method of converting passive immunity into active immunity can be used as a basis for therapy against cancer, disease, or infection or as an enhancement to current therapies against cancer, disease, or infection.

The step of increasing immune complex formation or immune complex number in the subject can comprise, for example, administering to the subject: (a) the antibody or antibody fragment such that the antibody or antibody fragment forms immune complexes with its target antigen in the subject, and/or (b) immune complexes that comprise the antibody or antibody fragment and the target antigen. The antibody, antibody fragment, and/or immune complexes are co-administered with the agent in an amount effective to induce, activate, or stimulate T helper and or T cytotoxic cells that have T cell receptors specific to the target antigen in the subject.

The methods of the invention are applicable for subjects who are afflicted with cancer, a pathogenic infection, disorder, or disease.

The cancer can be, for example, B cell lymphoma, colon cancer, lung cancer, renal cancer, bladder cancer, T cell lymphoma, myeloma, leukemia, chronic myeloid leukemia, acute myeloid leukemia, chronic lymphocytic leukemia, acute lymphocytic leukemia, hematopoietic neoplasias, thymoma, lymphoma, sarcoma, lung cancer, liver cancer, non-Hodgkins lymphoma, Hodgkins lymphoma, uterine cancer, renal cell carcinoma, hepatoma, adenocarcinoma, breast cancer, pancreatic cancer, liver cancer, prostate cancer, head and neck carcinoma, thyroid carcinoma, soft tissue sarcoma, ovarian cancer, primary or metastatic melanoma, squamous cell carcinoma, basal cell carcinoma, brain cancer, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, astrocytoma, medulloblastoma, craniopharyngioma, ependymoma, pinealoma, hemangioblastoma, acoustic neuroma, oligodendroglioma, meningioma, retinoblastoma, leukemia, melanoma, neuroblastoma, small cell lung carcinoma, bladder carcinoma, lymphoma, multiple myeloma, and medullary carcinoma.

The pathogenic infection can be caused by, for example, a bacterium, parasite, virus, fungus, or protozoa.

The T regulatory cell inhibitory agent (Treg agent) can be, for example, ONTAK, HuMax-Tac, Zenapax, or MDX-010 or a combination thereof. The Treg agent can comprise an antibody, or a fragment thereof, which specifically binds to a T regulatory cell surface protein. The T regulatory cell surface protein can be, for example, CD25 or CTLA4. The antibody, or fragment thereof, can further comprise a radionuclide or toxic moiety such that the antibody can kill the T regulatory cell. Antibodies that comprise a Treg agent can target a surface protein of the Treg cell, which include, for example, CD25, CD4, CD28, CD38, CD62L (selectin), OX-40 ligand (OX-40L), CTLA4, CCR4, CCR8, FOXP3, LAG3, CD103, NRP-1, or glucocorticoid-induced TNF receptor (GITR). The Treg agent can comprise a fusion protein, and the fusion protein can comprise a targeting moiety and a toxic moiety. The targeting moiety can comprise a ligand or portion thereof of a regulatory T cell surface protein. The ligand can be, for example, IL2, T cell receptor (TCR), MHCII, CD80, CD86, TARC, CCL17, CKLF1, CCL1, TCA-3, eotaxin, TER-1, E-cadherin, VEGF, semaphorin3a, CD134, CD31, CD62, CD38L, or glucocorticoid-induced TNF receptor ligand (GITRL). The toxic moiety can comprise, for example, lectin, ricin, abrin, viscumin, modecin, diphtheria toxin, cholera toxin, gelonin, Pseudomonas exotoxin, Shigella toxin, botulinum toxin, tetanus toxin, calicheamicin, or pokeweed antiviral protein.

The target antigen of the methods of the invention can comprise a cancer or tumor antigen. A cancer or tumor antigen can comprise, for example, an antigen selected from HER2, BRCA1, prostate-specific membrane antigen (PSMA), MART-1/MelanA, prostatic serum antigen (PSA), squamous cell carcinoma antigen (SCCA), ovarian cancer antigen (OCA), pancreas cancer associated antigen (PaA), MUC-1, MUC-2, MUC-3, MUC-18, carcino-embryonic antigen (CEA), polymorphic epithelial mucin (PEM), Thomsen-Friedenreich (T) antigen, gp100, tyrosinase, TRP-1, TRP-2, NY-ESO-1, CDK-4, b-catenin, MUM-1, Caspase-8, KIAA0205, HPVE7, SART-1, SART-2, PRAME, BAGE-1, DAGE-1, RAGE-1, NAG, TAG-72, CA125, mutated p21ras, mutated p53, HPV16 E7, RCC-3.1.3, MAGE-1, MAGE-2, MAGE-3, MAGE-4, MAGE-11, GAGE-I, GAGE-6, GD2, GD3, GM2, TF, sTn, gp75, EBV-LMP 1, EBV-LMP 2, HPV-F4, HPV-F6, HPV-F7, alpha-fetoprotein (AFP), CO17-1A, GA733, gp72, p-HCG, gp43, HSP-70, p17 mel, HSP-70, gp43, HMW, HOJ-1, HOM-MEL-55, NY-COL-2, HOM-HD-397, HOM-RCC-1.14, HOM-HD-21, HOM-NSCLC-11, HOM-MEL-2.4, HOM-TES-11, melanoma gangliosides, TAG-72, prostatic acid phosphatase, protein MZ2-E, folate-binding-protein LK26, truncated epidermal growth factor receptor (EGFR), GM-2 and GD-2 gangliosides, polymorphic epithelial mucin, folate-binding protein LK26, pancreatic oncofetal antigen, cancer antigen 15-3, cancer antigen 19-9, cancer antigen 549, or cancer antigen 195.

The target antigen of the methods of the invention can comprise an antigen from a pathogen, such as a viral antigenic peptide or protein. The viral antigenic peptide or protein can be from a protein expressed by, for example, Arboviruses, Herpesviruses, herpes simplex viruses, Epstein Barr virus, cytomegalovirus, varicella-zoster virus, human herpes virus 6, human herpes virus 8, herpes B virus Hepadnaviruses, hepatitis virus A, B, C, D, E, F, or G, Togaviruses, Venezuelan equine encephalitis virus, Coronaviruses, severe acute respiratory syndrome virus, Picornaviruses, polioviruses, Flaviviruses, human hepatitis C virus, yellow fever virus, dengue viruses, Retroviruses, human immunodeficiency viruses, human T lymphotropic viruses, Paramyxoviruses, respiratory syncytial virus, Reoviruses, rotaviruses, Bunyaviruses, hantaviruses, Filoviruses, Ebola virus, Adenoviruses, Parvoviruses, parvovirus B-19; Papovaviruses, human papilloma viruses, Rhabdoviruses, rabies virus, Arenaviruses, Lassa virus, Orthomyxoviruses, influenza viruses, Poxviruses, Orf virus, molluscum contageosum virus, Canine distemper virus, Canine contagious hepatitis virus, Feline calicivirus, Feline rhinotracheitis virus, TGE virus, smallpox virus, Monkey pox virus, rhinoviruses, orbiviruses, picodnaviruses, encephalomyocarditis virus, Parainfluenza viruses, adenoviruses, Coxsackieviruses, Echoviruses, Rubeola virus, Rubella virus, human metapneuomovirus, enteroviruses, Foot and mouth disease virus, simian virus 5, or human parainfluenza virus type 2.

The pathogen antigen can comprise a bacterial antigenic peptide or protein. The bacterial antigen can be from a protein or peptide expressed by, for example, Mycoplasma sp., Ureaplasma sp., Neisseria sp., Treponema sp., Bacillus sp., Haemophilus sp., Rickettsia sp., Chlamydia sp., Corynebacterium sp., Mycobacterium sp., Clostridium sp., Legionella sp., Shigella sp., Salmonella sp., pathogenic Escherichia sp., Vibrio sp., Staphylococcus sp., Bordatella sp., Moraxella sp., Streptococcus sp., Campylobacter sp., Borrelia sp., Leptospira sp., Pseudomonas sp., Helicobacter sp., Erlichia sp., or Klebsiella sp.

The pathogen antigen can comprise a fungal antigenic peptide or protein. The fungal antigen can be from a protein or peptide expressed by, for example, by Aspergillus sp., Pneumocystis sp. (such as P. carinii), Tinea sp., Candida sp., Sporothrix sp., Cryptococcus sp., Histoplasma sp., or Coccidioides sp.

The pathogen antigen can comprise a parasitic or protozoal antigenic peptide or protein. The protozoan antigen can be from a protein or peptide expressed by, for example, by Trypanosoma sp., Endamoeba sp., Giardia sp., Plasmodium sp., Babeosis sp., Toxoplasma sp., or Leishmania sp. The parasitic antigen can be from a protein or peptide expressed by, for example, Schistosoma sp., Taenia sp., Echinococcus sp., Hymenolepsis sp., Diphyllobotrium sp., Fasciolopsis sp., Trichinella sp., or Ascaris sp.

The methods of the invention can further comprise administering a vaccine that comprises the target antigen. The methods of the invention can further comprise administering a chemotherapy drug, an antibiotic, an antifungal drug, an antiviral drug, anti-parasitic drug, or an anti-protozoal drug or a combination thereof.

The chemotherapy drug can be, for example, an alkylating agent, a nitrosourea, an anti-metabolite, a topoisomerase inhibitor, a mitotic inhibitor, an anthracycline, a corticosteroid hormone, a sex hormone, or a targeted anti-tumor compound or a combination thereof. The targeted anti-tumor compound can be, for example, imatinib (Gleevec), gefitinib (Iressa), erlotinib (Tarceva), rituximab (Rituxan), or bevacizumab (Avastin). The alkylating agent can be, for example, busulfan, cisplatin, chlorambucil, cyclophosphamide (Cytoxan), dacarbazine (DTIC), mechlorethamine, melphalan, or temozolomide. The anti-metabolite can be, for example, 5-fluorouracil or methotrexate. The topoisomerase inhibitor can be, for example, topotecan, etoposide, or teniposide. The anthracycline can be, for example, daunorubicin, doxorubicin, epirubicin, idarubicin, or mitoxantrone.

### BRIEF DESCRIPTION OF THE DRAWINGS

FIGS. 1A-1D depict graphs showing tumor size over the course of 30 days in mice that were immunized with HER-2 immune complexes (ICs) following regulatory T cell inhibition. The graphs show that the mice are protected from challenge with HER-2 expressing tumors. FIG. 1A shows the tumor growth with no treatment (control). FIG. 1B shows tumor growth with only treatment with ONTAK. FIG. 1C shows the tumor size with treatment of only HER-2 IC. The figure demonstrates that intravenous injection of HER-2 ICs alone does not provide effective tumor protection. Only the combination of HER-2 IC and ONTAK results in inhibition of the regulatory T cells and a positive result as to the tumor growth. As shown in FIG. 1D, immunization with HER-2 ICs and ONTAK resulted in tumor rejection in 6 of 7 mice and the effect is synergistic rather than additive as neither treatment when given alone provided significant protection.

FIGS. 2A-2B show bar graphs depicting responses observed from stimulated splenocytes in mice. The mice were immunized with HER-2 ICs in the absence of regulatory T cells and the results show an induction of CD4 (FIG. 2A) and CD8 (FIG. 2B) anti-HER2 effector T cells.

### DETAILED DESCRIPTION OF THE INVENTION

The present invention provides methods for converting passive immunity (for example, provided by antibody administration or vaccination) into active immunity. In certain embodiments, the invention converts passive immunity to active immunity through two events: (1) increasing immune complex number or formation in a subject (wherein the immune complex comprises a target antigen bound to an immunoglobulin molecule having at least a variable domain specific to the target antigen and an Fc receptor binding region), and (2) depleting or decreasing the population of T regulatory cells in the subject or decreasing/inhibiting T regulatory cell function in the subject.

Immune complex number can be increased in the subject, for example, by administering pre-formed immune complexes to the subject or by administering antibodies specific to a desired target antigen(s) to the subject such that the antibodies will likely form immune complexes in the subject. The importance of increasing immune complex number/formation for inducing active immunity from passive immunity can be based, at least in part, on the role of antigen presenting cells. APCs express Fc receptors that bind to immune complexes, whereupon binding, the immune complexes are internalized by the APC and the antigen in the immune complex is processed and presented by MHC molecules on the surface of the APC. T-cells that express TCR's specific to the MHC-antigen complex are activated by the APC such that active immunity to the antigen begins to be acquired. However, as shown herein, administration of immune complexes or antibodies alone is not sufficient to induce an effective active immune response against a particular antigen. In other words, administration of immune complexes or antibodies or antigens alone is not sufficient to convert these administrations of passive immunity into active immunity. Rather, the invention has determined that to induce an effective active immune response against a particular antigen from a method of passive immunity, the method needs to inhibit T regulatory cell function (or deplete/decrease T regulatory cells in the subject) in combination with increasing immune complex number (where the immune complex comprises an antigen of the target disease, disorder, or microorganism) in the subject.

A method for converting protective immunity to active immunity against an antigen comprises administering an effective amount of an agent to a subject, wherein the agent decreases the activity or function of regulatory T cells (Tregs) in the subject and/or wherein the agent substantially depletes the Treg population temporarily in the subject, in combination with an antibody that is specific to a target antigen or an immunoreactive fragment thereof (wherein the antibody or fragment thereof is capable of binding to an Fc receptor). The target antigen can be, for example, a tumor antigen, a pathogen antigen, or a disease antigen. Thus, depending upon the target antigen, the methods of the invention can be used in the treatment of cancer, infection, or disease.

The method can further comprise administering a chemotherapy drug, an antibiotic, an antifungal drug, an antiviral drug, anti-parasitic drug, or an anti-protozoal drug or a combination thereof.

Terms

As used herein, the term “passive immunity” includes the situation where temporary immunity to a specific infection, disease or disorder can be induced in a subject by providing the subject with externally produced immune molecules, known as antibodies or immunoglobulins.

As used herein, the term “active immunity” refers to the events of adaptive immunity as opposed to innate immunity or passive immunity. The adaptive immune response or adaptive immunity is the response of antigen specific lymphocytes to antigen, including the development of immunological memory. Adaptive immune responses are generated by clonal selection of lymphocytes. Adaptive immune responses are also known as acquired immune responses. Thus, active immunity refers to processes of adaptive or acquired immunity in which recognition of a foreign or disease antigen triggers a series of coordinated cellular events (for example, antigen presenting cells present the antigen to activate T cells, which T cells in turn release cytokines to coordinate/stimulate more T cells and other effector cells and/or B cells) that can result in effector cells of the immune system to attack infected/diseased cells and in B cells producing antibodies specific to the antigens (such as tumor antigens, antigens of pathogens, etc.). For the purposes of the invention, conversion of passive immunity to active immunity can comprise the induction of antigen-specific effector T cell responses (see Examples).

The term “co-administration” refers to the combined administration of antibodies/immune complexes with a Treg agent. The co-administration does not have to be exactly contemporaneous in time, i.e., in the same injection. Rather, co-administration should at least comprise the administration of antibodies/immune complexes soon before or after the Treg agent(s) have an inhibitory effect on T regulatory cell function or viability.

The terms “Treg agent” or “Treg inhibitor” herein refer to an agent that: (1) inhibits or decreases the activity or function of a regulatory T cell, (2) decreases the population of regulatory T cells in a subject (in one embodiment, the decrease can be temporary, for example, for a few hours, a day, a few days, a week, or a few weeks), or (3) substantially ablates or eliminates the population of regulatory T cells in a subject (in one embodiment, the ablation or elimination can be temporary, for example, for a few hours, a day, a few days, a week, or a few weeks). A Treg inhibitor can decrease the suppression of immune system activation and can decrease prevention of self-reactivity. Exemplary Treg inhibitors may include, but are not limited to, a compound, antibody, fragment of an antibody, or chemical that targets a Treg cell surface marker (such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1). In certain embodiments, a Treg inhibitor targets a Treg cell surface marker that is involved in Treg activation such that the Treg inhibitor prevents Treg activation. A Treg inhibitor may include, but is not limited to, antibodies, fusion proteins, ONTAK, HuMax-Tac, Zenapax, or MDX-010, aptamers, siRNA, ribozymes, antisense oligonucleotides, and the like. The administration of a Treg inhibitor or derivatives thereof can block the action of its target, such as a Treg cell surface marker. A Treg inhibitor can have an attached toxic moiety such that upon internalization of the inhibitor, the attached toxic moiety can kill the T regulatory cell.

As used herein, the terms “antibody” and “antibodies” refer to monoclonal antibodies, multispecific antibodies, human antibodies, polyclonal antibodies, humanized antibodies, chimeric antibodies, single-chain Fvs (scFv), single chain antibodies, single domain antibodies, Fab fragments, F(ab) fragments, disulfide-linked Fvs (sdFv), anti-idiotypic (anti-Id) antibodies, and epitope-binding fragments of any of the above. Antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

A typical antibody contains two heavy chains paired with two light chains. A full-length heavy chain is about 50 kD in size (approximately 446 amino acids in length), and is encoded by a heavy chain variable region gene (about 116 amino acids) and a constant region gene. There are different constant region genes encoding heavy chain constant region of different isotypes such as alpha, gamma (IgG₁, IgG₂, IgG₃, IgG₄), delta, epsilon, and mu sequences. A full-length light chain is about 25 Kd in size (approximately 214 amino acids in length), and is encoded by a light chain variable region gene (about 110 amino acids) and a kappa or lambda constant region gene. The variable regions of the light and/or heavy chain are responsible for binding to an antigen, and the constant regions are responsible for the effector functions typical of an antibody. In certain embodiments, an antibody administered to a subject for the purpose of increasing immune complex number in the subject requires that the antibody comprise a portion of a constant region that binds to an Fc receptor.

As used herein, the term “toxic moiety” includes naturally occurring (as well as derivatized or chemically modified forms thereof) or synthetic molecules or moieties that are proteinaceous or non-proteinaceous and that are toxic to cells, such as Treg cells. “Toxic moieties” include, for example, portions of naturally occurring toxins that retain the property of toxicity (such as toxic moieties (e.g., A chains) of bipartate toxins). The term “toxic moiety” also can include antibiotic molecules or other agents (e.g. chemotherapeutic agents) that have cellular cytotoxic effects. Toxic moieties bring about the death of cells by any of a variety of mechanisms, e.g., by acting on cellular machinery after internalization into the cell or by forming holes in cellular membranes. Non-limiting examples of toxic moieties described herein include lectins (such as ricin, abrin, viscumin, modecin, and the like), diphtheria toxin, cholera toxin, gelonin, Pseudomonas exotoxin, Shigella toxin, botulinum toxin, tetanus toxin, calicheamicin, and pokeweed antiviral protein.

A “derivative” refers to a molecule that shares substantial structural similarity to its parent molecule. A protein derivative encompasses a protein, which includes a change to its amino acid sequence and/or chemical quality of the amino acid e.g., amino acid analogs, when compared to its parent protein. For example, in the context of a protein molecule (e.g., proteins, polypeptides, and peptides, such as antibodies), “derivative” refers to a protein molecule that comprises an amino acid sequence that has been altered by the introduction of amino acid residue substitutions, deletions, and/or additions. The term “derivative” as used herein also refers to a protein molecule that has been modified, for example, by the covalent attachment of any type of molecule to the protein molecule. A derivative of a protein molecule may be produced by chemical modifications using techniques known to those of skill in the art.

As used herein, a “fragment” or “portion” is any part or segment of a molecule. For example, a fragment of a molecule can be a part that recognizes and binds its natural target (such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1). In the case of an antibody, the fragment can be a binding portion of the whole antibody.

“Effective amount” means the amount of a therapeutic substance or composition which is sufficient to reduce (to any extent) or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). An “effective amount” does not require there be a cure of a disorder or symptom. The effective amount will vary with the age, gender, race, species, general condition, etc., of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (for example, see Gennaro et al., Eds. Remington's The Science and Practice of Pharmacy 20^{th} edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.).

The terms “prevent,” “preventing,” and “prevention” refer herein to the inhibition of the development or onset of a disorder or the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, the term “fusion protein” refers to a polypeptide or protein (including, but not limited to an antibody) that comprises an amino acid sequence of a first protein or polypeptide or functional fragment, analog or derivative thereof, and an amino acid sequence of a heterologous protein, polypeptide, or peptide (i.e., a second protein or polypeptide or fragment, analog or derivative thereof different than the first protein or fragment, analog or derivative thereof). A fusion protein can comprise a prophylactic or therapeutic agent fused to a heterologous protein, polypeptide or peptide. Accordingly, the heterologous protein, polypeptide or peptide may or may not be a different type of prophylactic or therapeutic agent. For example, two different proteins, polypeptides or peptides with immunomodulatory activity may be fused together to form a fusion protein. Fusion proteins may retain or have improved activity relative to the activity of the original protein, polypeptide or peptide prior to being fused to a heterologous protein, polypeptide, or peptide.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey, such as a cynomolgous monkey, a chimpanzee, and a human), and most preferably a human. The subject can be a non-human animal such as a bird (e.g., a quail, chicken, or turkey), a farm animal (e.g., a cow, horse, pig, or sheep), a pet (e.g., a cat, dog, or guinea pig), or laboratory animal (e.g., an animal model for a disorder). If the subject is a human, it can be an infant, child, adult, or senior citizen.

“Treat,” “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay in the progression of the condition, prevention or delay of the onset of the disorder, and/or change in clinical parameters, disease or illness, etc., as would be well known in the art.

Passive and Active Immunity

In certain embodiments, the invention provides methods for the induction of an active antigen specific immune response from passive immunization coupled with Treg inhibition/ablation. Passive immunization can comprise administration of antibodies or immune complexes. In other embodiments, the methods of the invention can be used to enhance vaccination—for example, passive immunization with antibodies against a specified antigen(s) is converted to active immunity against the antigen when passive immunization is coupled with Treg agent(s)—this can help to enhance the effects of subsequent or prior vaccination with the same specified antigen(s).

The subject according to the invention can be an animal, such as a mammal. The mammal can be a non-primate, such as domestic animals, commercial animals, farm animals, and the like (for example, a cow, pig, bird, sheep, goat, horse, cat, dog, rat, rabbit, mouse, and the like) or a primate (for example, a monkey, such as a cynomolgous monkey, a chimpanzee, a human). Non-limiting representative subjects according to the invention may be a human infant, a pre-adolescent child, an adolescent, an adult, or a senior/elderly adult.

Passive immunity can be acquired naturally or artificially. For example, naturally acquired passive immunity occurs during pregnancy, in which certain antibodies are passed from the mother to the fetus via the bloodstream. Artificially acquired passive immunity is a temporary immunity against an antigen provided for by immunization via injection of antibodies that are not produced by the cells of the receiving subject. Naturally acquired active immunity occurs when the subject is exposed to a live pathogen. The subject subsequently develops the disease and becomes immune due to the primary immune response. An individual can artificially acquire active immunity via a vaccine that contains an antigen to a disease, infection, disorder, and the like, wherein the vaccine stimulates a primary response against the antigen without causing symptoms of the disease, disorder, etc.

Artificially acquired passive immunity is short-term provision of antibodies via injection of antibodies that are not produced by the cells of the receiving subject. The current methods of the invention present the novelty of the idea of converting passive immunotherapy with antibodies into an active immunization protocol via inhibition of T regulatory cell function coupled with increasing the number of immune complexes comprising a target antigen(s) (i.e., target antigens of the active immune response).

The methods of the invention may utilize various classes of therapeutics. For example, methods can comprise administering: (1) an agent that inhibits Tregs (such as those previously described) via targeting Treg cell surface markers (such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1) and (2) an anti-tumor or anti-pathogen antibody or immunoreactive fragment thereof (such as an immunoreactive single chain antibody or an antibody directed against a pathogenic antigen or an anti-tumor antibody, or a preformed immune complex (IC)) that comprises an FcR binding region.

These antibodies or fragments thereof can be obtained commercially, custom generated, or synthesized against an antigen of interest according to methods established in the art as described above. A pathogenic antigen can be a protein or polypeptide expressed by a foreign virus or microorganism (such as a bacterium, fungus, protozoan, or parasite and the like) that is introduced into a subject (for example, an animal, such as a human) wherein such a pathogenic entity can cause the infection.

Non-limiting examples of pathogenic entities include prokaryotic pathogens (e.g., Rickettsia sp., Pseudomonas sp., Mycoplasma sp., Mycobacterium sp., Neisseria sp., Legionella sp., Chlamydia sp., and the like); fungal pathogens (for example, Candida sp.); protozoal pathogens (e.g., Leishmania sp. and Trypanosoma sp.) and viral pathogens (such as echovirus, rotavirus, lentivirus, hepatitis type A, hepatitis type B, hepatitis type C, adenovirus, herpesvirus, rhinovirus, arbovirus, measles virus, retrovirus, respiratory syncytial virus (RSV), hantavirus, coxsackie virus, mumps virus, papilloma virus, influenza, varicella, papova virus, cytomegalovirus, coronavirus, rubella virus, polio virus, human immunodeficiency virus type I (HIV-I), and human immunodeficiency virus type II (HIV-II)).

The antibodies used for increasing IC number in a subject, for example, can be specifically directed again pathogenic antigens. The pathogenic antigen of this invention can be a viral antigenic peptide or protein that includes, but is not limited to, antigens from Arboviruses; Herpesviruses including herpes simplex viruses (HSV-1 and HSV-2), Epstein Barr virus (EBV), cytomegalovirus (CMV), varicella-zoster virus (VZV), human herpes virus 6 (HHV-6), human herpes virus 8 (HHV-8), and herpes B virus; Hepadnaviruses including hepatitis A, B, C, D, E, F, G, etc. (e.g., HBsAg, HBcAg, HBeAg); Togaviruses including Venezuelan equine encephalitis virus; Coronaviruses including corona viruses such as the severe acute respiratory syndrome (SARS) virus; Picornaviruses including polioviruses; Flaviviruses including human hepatitis C virus (HCV), yellow fever virus and dengue viruses; Retroviruses including human immunodeficiency viruses (HIV) (e.g., gp120, gp160, gp41, an active (i.e., antigenic) fragment of gp120, an active (i.e., antigenic) fragment of gp160 and/or an active (i.e., antigenic) fragment of gp41) and human T lymphotropic viruses (HTLV1 and HTLV2); Paramyxoviruses (for example, mumps antigens) including Morbillivirus sp. (for example, measles antigens) and respiratory syncytial virus; Reoviruses including rotaviruses; Bunyaviruses including hantaviruses; Filoviruses including Ebola virus; Adenoviruses; Parvoviruses including parvovirus B-19; Papovaviruses including human papilloma viruses; Rhabdoviruses including rabies virus; Arenaviruses including Lassa virus; Orthomyxoviruses including influenza viruses (e.g., NP, HA antigen); Poxviruses including Orf virus, molluscum contageosum virus, Canine distemper virus, Canine contagious hepatitis virus, Feline calicivirus, Feline rhinotracheitis virus, TGE virus (swine), smallpox virus and Monkey pox virus; rhinoviruses; orbiviruses; picodnaviruses; encephalomyocarditis virus (EMV); Parainfluenza viruses, adenoviruses, Coxsackieviruses, Echoviruses, Rubeola virus, Rubella virus, human papillomaviruses, human metapneuomovirus, enteroviruses, Foot and mouth disease virus, simian virus 5, human parainfluenza virus type 2, and any other pathogenic virus known in the art (see, e.g., Fields et al., Eds., Fundamental Virology, 3^{rd} ed., Lippincott-Raven, New York, 1996; the entire contents of which are incorporated by reference herein for the teachings of pathogenic viruses).

In addition, the pathogenic antigen can be an antigenic peptide or protein of a pathogenic microorganism (such as Gram-negative and Gram-positive bacteria), which can include but is not limited to, Mycoplasma sp., Ureaplasma sp., Neisseria sp., Treponema sp., Bacillus sp., Haemophilus sp., Rickettsia sp., Chlamydia sp., Corynebacterium sp. (for example diphtheria toxin or other diphtheria antigens of Corynebacterium sp., such as C. diphtheriae), Mycobacterium sp., Clostridium sp. (for example, tetanus toxin and other tetanus antigens of C. tetani), Legionella sp., Shigella sp., Salmonella sp., pathogenic Escherichia sp. (for example, E. coli), Vibrio sp., Staphylococcus sp., Bordatella sp. (for example, pertussis toxins of Bordetella sp., such as B. pertussis), Moraxella sp., Streptococcus sp., Campylobacter sp., Borrelia sp., Leptospira sp., Pseudomonas sp., Helicobacter sp., Erlichia sp., Klebsiella sp., and any other pathogenic microorganism known in the art (see, e.g., Tortora et al., Eds., (2002) Microbiology an Introduction, 7^{th} ed., Benjamin Cummings, San Francisco Calif.; the entire contents of which are incorporated herein by reference for the teachings of pathogenic microorganisms).

Antigens of this invention that are part of an IC also can be antigenic peptides or proteins from pathogenic protozoa or parasites, that include, but are not limited to, Endamoeba sp., Schistosoma sp., Taenia sp., Diphyllobotrium sp., Schistosoma sp., Fasciolopsis sp., Trichinella sp., Ascaris sp., Trypanosoma sp., Echinococcus sp., Hymenolepsis sp., Plasmodium sp. (for example, malaria antigens), Giardia sp., Babesia sp., Toxoplasma sp., Leishmania sp., and any other protozoan or parasitic pathogen known in the art (see, e.g., Tortora et al., Eds., (2002) Microbiology an Introduction 7^{th} ed., Benjamin Cummings, San Francisco Calif.; the entire contents of which are incorporated herein by reference for the teachings of pathogenic microorganisms).

Furthermore, IC antigens can also be antigenic peptides or proteins from pathogenic yeast and fungi, which include, but are not limited to, Aspergillus sp., Pneumocystis sp. (such as P. carinii), Tinea sp., Candida sp., Sporothrix sp., Cryptococcus sp., Histoplasma sp., Coccidioides sp., and any other pathogenic fungus known in the art (see, for example, Tortora et al., Eds., (2002) Microbiology an Introduction 7^{th} ed., Benjamin Cummings, San Francisco Calif.; the entire contents of which are incorporated herein by reference for the teachings of pathogenic microorganisms).

In the present invention, passive immunotherapy is converted into active immunization. In one embodiment, the use of antibodies or fragments thereof directed at Treg cell surface markers in combination with an antibody or fragment thereof directed at a pathogenic antigen (such as those described above) induces or increases effector cell responses against the pathogen having the antigen. In another embodiment, the use of antibodies or fragments thereof directed at Treg cell surface markers in combination with an antibody or fragment thereof directed at a tumor antigen induces or increases effector cell responses against the tumor having the antigen. Such treatments convert passive immunity (i.e., administration of antibodies against pathogens/tumors alone) into active immunotherapy (i.e., as indicated by responses showing acquired immunity, for example, greater effector cell responses against the pathogen/tumor, and/or production of greater affinity antibodies (hypermutation)).

According to methods of the invention, antibodies or fragments thereof directed to a tumor antigen can be an immunoreactive fragment of an antibody. This peptide can be obtained commercially, custom generated, or synthesized against an antigen of interest according to methods established in the art as described above. A tumor antigen can be a protein or polypeptide expressed by a tumorigenic cell.

In one embodiment, the anti-tumor antibody is an antibody or fragment thereof that binds to a tumor cell specific protein, such as anti-tumor antigens. Some non-limiting examples of anti-tumor antigens include HER2, BRCA1, prostate-specific membrane antigen (PSMA), MART-1/MelanA, prostatic serum antigen (PSA), squamous cell carcinoma antigen (SCCA), ovarian cancer antigen (OCA), pancreas cancer associated antigen (PaA), MUC-1, MUC-2, MUC-3, MUC-18, carcino-embryonic antigen (CEA), polymorphic epithelial mucin (PEM), Thomsen-Friedenreich (T) antigen, gp100, tyrosinase, TRP-1, TRP-2, NY-ESO-1, CDK-4, β-catenin, MUM-1, Caspase-8, KIAA0205, HPVE7, SART-1, SART-2, PRAME, BAGE-1, DAGE-1, RAGE-1, NAG, TAG-72, CA125, mutated p21ras, mutated p53, HPV16 E7, RCC-3.1.3, MAGE-1, MAGE-2, MAGE-3, MAGE-4, MAGE-11, GAGE-I, GAGE-6, GD2, GD3, GM2, TF, sTn, gp75, EBV-LMP 1, EBV-LMP 2, HPV-F4, HPV-F6, HPV-F7, alpha-fetoprotein (AFP), CO17-1A, GA733, gp72, p-HCG, gp43, HSP-70, p17 mel, HSP-70, gp43, HMW, HOJ-1, HOM-MEL-55, NY-COL-2, HOM-HD-397, HOM-RCC-1.14, HOM-HD-21, HOM-NSCLC-11, HOM-MEL-2.4, HOM-TES-11, melanoma gangliosides, TAG-72, prostatic acid phosphatase, protein MZ2-E, folate-binding-protein LK26, truncated epidermal growth factor receptor (EGFR), GM-2 and GD-2 gangliosides, polymorphic epithelial mucin, folate-binding protein LK26, pancreatic oncofetal antigen, cancer antigen 15-3, cancer antigen 19-9, cancer antigen 549, and cancer antigen 195.

For example, in the case of a subject afflicted with cancer, a Treg agent can be administered in combination with an anti-tumor antibody (or with an immune complex comprising the anti-tumor antibody and the tumor antigen to which it binds), and optionally with low doses of a chemotherapy drug (for example, those drugs listed below, such as cytoxan). The chemotherapeutic drug should be administered at a low dose wherein regulatory T cells are sensitive but which acts as a suboptimal dosing for tumor cells (Ghiringhelli, et al., (2004) Eur J Immunol 34(2):336-44).

Immune Complexes and Conversion of Passive to Active Immunity

Immune complexes comprise an antibody or fragments thereof directed at a specific antigen bound to its respective antigen, wherein the antigen can be a cancer/tumor antigen or a pathogenic antigen. The immune complexes can be generated in vitro (“pre-formed IC”) and subsequently be administered to a subject. In certain embodiments, the immune complexes comprise at least one immunoglobulin FcR binding region.

Immune complexes (ICs) can gain entry to APCs, including dendritic cells, through Fc receptor-mediated endocytosis and phagocytosis. Immune complex-loaded dendritic cells recently have been shown by to efficiently present antigens to T cells in vivo, yet the induction of T effector responses is not as robust as expected. This response might be due to concomitant induction of T regulatory cell activation.

Antibodies can bind activating or inhibitory Fc receptors and complement receptors on antigen-presenting cells with diverse consequences. For example, interactions on complement receptors can induce T cell tolerance. However, interactions with inhibitory Fc receptors also can induce T cell tolerance. Additionally, interactions with activating Fc receptors can induce T cell immunity (Karlsson et al., (1999) Proc Natl Acad Sci USA 96(5):2244-9; Karlsson et al., (2001) J Immunol 167(10): 5558-64; Wenersson et al., (2000) Scan J Immunol 52(6): 563-9; Jacquemin et al., (1995) Int Arch Allergy Immunol 107(1-3):313-5; Miescher et al., (2005) Blood 106(4): 1503-4; Miescher et al., (2004) Blood 103(11): 4028-35; Siragam et al. (2006) Nat Med. 12(6):688-692). Thus, immune complex delivery via an Fc receptor is a non-conventional approach with data on both sides arguing for both immunostimulatory and inhibitory effects (Karlsson et al., (1999) Proc Natl Acad Sci USA 96(5):2244-9; Karlsson et al., (2001) J Immunol 167(10): 5558-64; Wenersson et al., (2000) Scan J Immunol 52(6): 563-9; Jacquemin et al., (1995) Int Arch Allergy Immunol 107(1-3):313-5; Miescher et al., (2005) Blood 106(4): 1503-4; Miescher et al., (2004) Blood 103(11): 4028-35; Siragam et al. (2006) Nat Med. 12(6):688-692). Evidence that the Treg inhibition can interfere with these negative regulatory pathways has not previously been provided.

Passive administration of anti-tumor antibodies is not optimal. Such an administration path would potentially result in a minimal amount of tumor antigen:antibody immune complexes being available for Fc receptor (FcR)-mediated binding by dendritic cells. Thus a non-therapeutic amount of anti-tumor immune complexes would be processed. The current invention provides that these therapeutics (for example an anti-tumor antibody or an antibody directed at a pathogenic antigen in addition to an agent that decreases the activity of Tregs) can be used together to increase effector T cells, which can result in T cell proliferative responses (see Examples 1 and 2).

Data from Examples 1 and 2 shows that immune complexes alone fail to induce T cell effector responses by themselves. However, when Tregs are inhibited, T cell effector responses are induced. Thus, an active or protective T cell response can be generated. In one embodiment, Treg activity or function is decreased via treatment with a Treg agent. A Treg agent targets a Treg cell surface marker (such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1) and can encompass antibodies, fusion proteins, ONTAK, HuMax-Tac, Zenapax, or MDX-010, aptamers, siRNA, ribozymes, antisense oligonucleotides, and the like.

In the present invention, the use of antibodies directed against Treg cell surface markers in combination with an anti-tumor antibody is provided in order to decrease the function or activity of Tregs and subsequently enhance the anti-tumor response. In one embodiment, antibodies (such as polyclonal, monoclonal, humanized, and the like) are directed at CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1. Additionally, the method of the above invention provides for a new use of FDA-approved drug(s) that have the ability to inhibit regulatory T cells (for example, via decreasing the function or activity of Tregs). In another embodiment, ONTAK (monoclonal antibody that binds to the CD25 subunit of the IL-2 receptor), HuMax-Tac, Zenapax, or MDX-010, which is monoclonal antibodies directed against CTLA4, to be used.

The above-mentioned treatment combinations decrease the function or activity of Tregs in a subject (for example an animal, such as a human), which in turn, enhance tumor responses and the subsequent induction of effector T cell responses via anti-tumor antibody treatment. In one embodiment of the invention, an anti-tumor antibody can bind to an Fc receptor on the surface of a dendritic cell forming an IC whereby the IC is subsequently endocytosed via the Fc-mediated pathway. The dendritic cell then can display tumor antigens, which would activate T cells and thus lead to an attack of cancer cells expressing such antigens. But, ICs alone do not induce an effective T cell response.

Inhibition or Ablation of T Regulatory Cells

Enhancement of antigen presentation by APCs, including dendritic cells, can be exploited to induce active immunity to a specific external antigen (such as a pathogenic or tumor antigen). Fc receptor (FcR)-mediated targeting by an immune complex (IC) is one approach to enhance antigen presentation. However, as stated above, induced T-cell responses are limited in effector capacity. Inhibition of regulatory T-cells concomitant with vaccination was found to boost antibody-induced effector T cell responses, sufficient to impart tumor protection (see Examples 1 and 2).

In one embodiment, the invention provides a method for enhancing or inducing APC activation in a subject via using Treg agents that inhibit or decrease the activity or function of Tregs in order to promote the efficacy of chemotherapy drugs, antibiotics, antifungal drugs, antiviral drugs, anti-parasitic drugs, or anti-protozoal drugs and the like. The method comprises administering an effective amount of a Treg agent to a subject, wherein the Treg agent decreases the activity or function of Tregs, and administering an effective amount of an anti-tumor antibody or anti-pathogen antibody or an immunoreactive fragment thereof (in one embodiment, the antibody can be part of an immune complex with the tumor or pathogen antigen it binds to, such that the immune complex is administered) that contains an Fc receptor binding region, whereby the combination treatment results in enhancing or inducing APC activation in the subject. In addition, the method can further comprise administering a third therapeutic (such as a chemotherapy drug, an antibiotic, antifungal drug, antiviral drug, anti-parasitic drug, or anti-protozoal drug or a derivative thereof).

Treg agents can inhibit or decrease the activity or function of a regulatory T cell. Further, a Treg agent that is attached to a toxic moiety may be capable of depleting regulatory T cells in a subject. In certain embodiments, a Treg agent can target a Treg cell surface marker (such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1). CD4, CD25, CTLA-4 (cytotoxic T lymphocyte-associated antigen 4), and GITR (glucocorticoid-induced tumor necrosis factor receptor family-related gene) are cell surface marker molecules of regulatory T cells (J Allergy Clin Immun, (2002) 110: 693-701). CD4 is a marker for some thymic-derived populations of Tregs in addition to helper T cells. CD25 is a component of the IL-2 receptor, and can serve as a marker for activated T cells. GITR is strongly expressed on activated T cell and is weakly expressed overall on T cells during inactivation, dendritic cells, as well as macrophages (Nature Immunology, (2002) 3: 135). CD28, CCR4, CCR8, LAG3, and CD103 are also T cell markers. Additionally, FoxP3 (fork-head box protein 3) transcription factor, the master gene involved in the differentiation and functional expression of regulatory T cell, can be used as a Treg molecular marker (Science (2003) 299: 1057-61).

Further description and information on human CD4+CD25+ regulatory T cells may be found in the following references, which are all hereby incorporated by reference: Jonuleit et al. (2001) J Exp Med. 193:1285-94; Levings et al. (2001) J Exp Med 193:1295-1301; Dieckmann et al. (2001) J Exp Med 193:1303-1310; and Yamagiwa et al. (2001) J. Immunol. 166:7282-89, Stephens et al. (2001) Eur. J. Immunol. 31:1247-1254; and Taams et al. (2001) Eur. J. Immunol. 31:1122-1131.

The Treg agent or inhibitors of the invention can be used to decrease the activity or function of Tregs in the method of the invention (for example by blocking the action of its target, such as a Treg cell surface marker) can be any compound, small molecule, peptide, protein (such as antibodies), fusion protein, aptamer, RNAi, siRNA, or antisense oligonucleotide and the like.

For example, a Treg agent according to the invention can be a protein, such as an antibody (monoclonal, polyclonal, humanized, and the like), or a binding fragment thereof, directed against a Treg cell surface marker (for example, those listed above). An antibody fragment can be a form of an antibody other than the full-length form and includes portions or components that exist within full-length antibodies, in addition to antibody fragments that have been engineered. Antibody fragments include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed. Garland Publishing). In one embodiment, an antibody directed at a Treg cell surface protein, such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1, could be monoclonal or polyclonal.

In some embodiments, antibodies can also be commercially marketed drugs (such as Zenapax, HuMax-TAC, MDX-010, and the like). Other drugs that possess the ability to inhibit Tregs can be used according to the present invention. These drugs can also be humanized, polyclonal, or monoclonal antibodies directed against Treg cell surface markers, such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1, and the like. For example, Zenapax (Daclizumab) is known as an Interleukin-2 receptor inhibitor that prevents the body's immune system from responding to and rejecting a foreign antigen by blocking the receptor for Interleukin-2. Zenapax is an immunosuppressive, humanized IgG1 monoclonal antibody produced by recombinant DNA technology that binds specifically to the alpha subunit (p55 alpha, CD25, or Tac subunit) of the human high-affinity interleukin-2 (IL-2) receptor expressed on the surface of activated lymphocytes.

In another embodiment, the method can include the administration of a fully human antibody against human CTLA-4. CTLA-4 is a surface molecule on T cells responsible for suppressing the immune response, wherein the use of such an antibody to block CTLA-4 may enable the immune systems of cancer patients to more effectively fight tumors.

HuMax-TAC is a fully human monoclonal antibody that targets the TAC antigen. TAC is also known as CD25 or the alpha subunit of the interleukin-2 receptor (IL-2Rα) and is overexpressed by activated T-cells.

Additionally, a Treg agent can be a non-antibody peptide or polypeptide that binds to a Treg cell surface marker. A peptide or polypeptide can be a portion of a protein molecule of interest other than the full-length form, and includes peptides that are smaller constituents that exist within the full-length amino acid sequence of a protein molecule of interest. These peptides can be obtained commercially or synthesized via liquid phase or solid phase synthesis methods (Atherton et al., (1989) Solid Phase Peptide Synthesis: a Practical Approach. IRL Press, Oxford, England). For example, the Treg agent can be a peptide that interacts with a Treg cell surface marker. The peptide or protein-related Treg agents can be isolated from a natural source, genetically engineered, or chemically prepared. These methods are well known in the art.

Antibodies or fragments thereof in addition to non-antibody peptides or polypeptides that bind to a Treg cell surface marker can be conjugated with a radionuclide or binding moiety or toxic moiety, wherein one molecule (such as a Treg cell surface marker polypeptide) is joined covalently or non-covalently to a second molecule (such as an affinity label, fluorophore, or radiolabel, for example a radionuclide).

Generally, radionuclides suitable for use in peptide conjugates can include those having suitable emission properties to provide ablation of targeted Tregs in situ, while not unduly exposing the surrounding cells and tissues to damaging levels of irradiation. An ideal radionuclide for use in such therapeutic compositions is a relatively short-lived α-emitter, a γ-emitter, or a β-emitter that emits enough gamma irradiation to cause local destruction. Non-limiting examples of radionuclides include lutetium-177, iodine-131, iodine-125, and phosphorus-32 (γ-emitters); actinium-225, astatine-211, and bismuth-212 and bismuth-213 (α-emitters); iodine-123, copper-64, iridium-192, osmium-194, rhodium-105, rhodium-186, samarium-153, and yttrium-88, yttrium-90, and yttrium-91.

A binding moiety is a portion of a molecule that retains the ability to bind to a second molecule when other portions of the molecule are removed or modified or when the binding moiety is placed into a heterologous context. For example, a Treg cell surface marker polypeptide conjugated with a binding moiety (for example, with steptavidin, avidin, and the like) can be used to clear the subject of Tregs according to clearing methods practiced in the art (Hamblett et al., (2005) Bioconjug Chem. 16(1):131-8; Wilbur et al., (2004) Bioconjug Chem. 15(3):601-16; Boerman, O., et al., (2003) Nucl Med. 44(3):400-11. Rosebrough S., (1993) Pharmacol Exp Ther. 265(1):408-15; Rosebrough S., (1993) Nucl Med Biol. 20(5):663-8) Non-limiting examples of binding moieties include biotin, FLAG tag, streptavidin, histidine, maltose-binding protein, glutathione sepharose, or immunoglobulin.

A fusion protein to be used with this invention can be a protein or polypeptide comprising a first amino acid sequence that is a fragment of a protein or a whole protein linked or joined to a second amino acid sequence that can be a peptide, a fragment of a protein or a whole protein and wherein the first and second amino acid sequences are not linked or joined in the same way in nature. Fusion proteins can comprise a targeting moiety (such as a ligand to a Treg cell surface marker polypeptide, such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1, and the like; or a polypeptide of a Treg cell surface marker ligand, for example, IL2, T cell receptor (TCR), MHCII, CD80, CD86, TARC, CCL17, CKLF1, CCL1, TCA-3, eotaxin, TER-1, E-cadherin, VEGF, semaphorin3a, CD134, CD31, CD62, CD38L, or glucocorticoid-induced TNF receptor ligand (GITRL)) in addition to a toxic moiety (described below) or a binding moiety (as described above). Fusion proteins can be generated according to methods practiced in the art via fusing portions of Treg cell surface markers to IgG or diphtheria toxin (DT₃₈₈) (for example, IL2-Ig, CTLA4-Ig, IL2-, and the like).

For example, in order to produce a fusion protein comprising a ligand to a Treg cell surface marker, such as a fusion protein comprising IL2, MHCII, CD80, CD86, TARC, CCL17, CKLF1, CCL1, TCA-3, eotaxin, TER-1, E-cadherin, VEGF, semaphorin3a, CD134, CD31, CD62, CD38L, or glucocorticoid-induced TNF receptor ligand (GITRL)), the nucleotide sequence coding for the protein, or a functional equivalent, is inserted into an appropriate expression vector, for example, a vector which contains the necessary elements for the transcription and translation of the inserted coding sequence. The host cells or cell lines transfected or transformed with recombinant expression vectors can be used for a variety of purposes. These include, but are not limited to, large-scale production of the fusion protein.

Methods which are well known to those skilled in the art can be used to construct expression vectors containing a fusion coding sequence and appropriate transcriptional and/or translational control signals. These methods include in vitro recombinant DNA techniques, synthetic techniques and in vivo recombination/genetic recombination. (See, e.g., the techniques described in Sambrook et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y.). RNA capable of encoding a polypeptide may also be chemically synthesized (Gait, ed., 1984, oligonucleotide Synthesis, IRL Press, Oxford).

A variety of host-expression vector systems may be utilized to express a fusion protein coding sequence. These include, but are not limited to, microorganisms such as bacteria (e.g. E. coli, B. subtilis) transformed with recombinant bacteriophage DNA, plasmid DNA or cosmid DNA expression vectors containing a coding sequence; yeast (e.g. Saccharomyces, Pichia) transformed with recombinant yeast expression vectors containing a coding sequence; insect cell systems infected with recombinant virus expression vectors (e.g., baculovirus) containing a coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a coding sequence; or mammalian cell systems (e.g. COS, CHO, BHK, 293, 3T3 cells). The expression elements of these systems vary in their strength and specificities.

Depending on the host/vector system utilized, any of a number of suitable transcription and translation elements, including constitutive and inducible promoters, may be used in the expression vector. For example, when cloning in bacterial systems, inducible promoters such as pL of bacteriophage A, plac, ptrp, ptac (ptrp-lac hybrid promoter; cytomegalovirus promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of a the antigen coding sequence, SV40-, BPV- and EBV-based vectors may be used with an appropriate selectable marker.

A wide variety of toxic moieties are known in the art that can be conjugated to Treg cell surface markers or to Treg cell surface marker ligands, such as IL2, MHCII, CD80, CD86, TARC, CCL17, CKLF1, CCL1, TCA-3, eotaxin, TER-1, E-cadherin, VEGF, semaphorin3a, CD134, CD31, CD62, CD38L, or glucocorticoid-induced TNF receptor ligand (GITRL) (see Hertler and Frankel (1989) J. Clin. Oncol. 7:1932-1942). For example, toxic moieties may disrupt the cell membrane without internalization, toxic moieties may be internalized via a non-specific mechanism, or toxic moieties may be specifically internalized, for example, by direct interaction with specific receptor proteins on the cell. Toxic moieties for use according to the invention can be naturally occurring or synthetic. Toxic moieties may be proteinaceous or non-proteinaceous, e.g., oligosaccharides and can be plant-, fungus- or even bacteria-derived toxic moieties. Non-limiting examples of toxic moieties include: A chain toxic moieties, such as abrin A chain, particularly ricin A chain; ribosome inactivating proteins such as saporin or gelonin; α-sarcin; lectin; viscumin; modecin; cholera toxin; aspergillin; restrictocin; ribonucleases such as placental ribonuclease; acalyphin; jatrophin; curcin; crotin; phenomycin; neomycin; pertussis toxin; a porin protein, such as gonococcal PI porin protein; Shigella toxin; botulinum toxin; tetanus toxin; diphtheria toxin; Pseudomonas exotoxin; pokeweed antiviral protein; and calicheamicin (Jaracz et al., (2005) Bioorg Med Chem. 13(17):5043-54; Johannes et al., (2005) Gene Ther. 12(18):1360-8; Sandvig et al., (2005) Gene Ther. 12(11):865-72).

Ribosome inactivating proteins (RIPs) are able to directly inhibit the ribosomal translational machinery. The heterodimer peptide ricin, such a toxic moiety, is derived from the castor bean plant (Ricinus communis). The toxic activity of ricin is found entirely in one of its subunits (ricin A-chain) and is thought to deactivate ribosome function by specifically depurinating the single adenine at position 4324 of 28S rRNA (Chen et al. (1998) Biochemistry 37:11605; Koehler et al. (1994) Bone Marrow Transplant 13:571-575; Duke-Cohan et al. (1993) Blood 82:2224-34). Another RIP toxic moiety is abrin, which is derived from the jequirity bean (Abrus precatorius). It is known to deactivate protein translation by the same mechanism as ricin-A (Krupakar et al. (1999) Biochem. J. 338:273-279). Other RIPs which can be used according to the invention include the plant cytotoxins saporin and gelonin. The Shiga-A toxic moiety from the microorganism Shigella dysenteriae also functions as an RIP (Fraser, M. E. (1994) Nat. Structural Biol. 1:59-64), as does the sarcin-A toxic moiety, derived from the mold Aspergillus giganteus (Lacadena et al. (1999) Proteins 37:474-484). Antibody-toxic moiety conjugates, which include ricin-A and similar toxic moieties, have been described previously in U.S. Pat. Nos. 4,590,017, 4,906,469, 4,919,927, and 5,980,896.

Toxic moieties involved in ADP-ribosylation of the elongation factor 2 (EF-2), such as, bacterial diphtheria toxin (from Corynebacterium diphtheriae) and/or in inhibition of protein synthesis (Foley et al. (1995) J. Biol. Chem. 270:23218-23225) can also be used according to the invention. Antibody-toxic moiety conjugates which include diphtheria toxin or related toxic moieties which ADP-ribosylate EF-2 have been described previously, e.g., in U.S. Pat. Nos. 4,545,985.

Other toxic moieties can also be utilized that bring about eukaryotic cell death via interfering with microtubule function. This results in mitotic arrest (Iwasaki (1998) Yakugaku Zasshi 118:112-126). One non-limiting example of these toxic moieties is the maytansinoid compounds (Takahashi et al. (1989) Mol. Gen. Genet. 220:53-59), which are found in certain mosses, for example Maytenus buchananii (see Larson et al. (1999) J. Nat. Prod. 62:361-363). Antibody-toxic moiety conjugates, which include maytansinoids, have been described previously in U.S. Pat. No. 5,208,020.

Additionally, other toxic moieties are able to activate the adenylate cyclase cAMP system, causing unregulated transport of anions and cations through the cell membranes. An example of this type of toxic moiety is the cholera toxin (de Haan et al. (1998) Immunol. Cell Biol. 76:270-279) derived from Vibrio cholerae, a microorganism that can cause fluid secretion and hemorrhage of intestinal cells.

The bacterial pertussis toxin (derived from Bordetella pertussis) is able to specifically target the eukaryotic G protein complex. The heterotrimeric G protein is a key element in the transduction of many extracellular signaling pathways, including those triggered by cytokine and hormone receptors. The pertussis toxin can ADP-ribosylate a subunit of the G protein complex, causing an uncoupling of its regulatory activity (Locht and Antoine (1995) Biochimie 77:333-340).

Ligands to Treg cell surface markers, as described above, can be fused to a toxic moiety in order to target Treg cells so that Treg activity and/or function can be inhibited or such that the Treg cell population can be substantially ablated/eliminated (temporarily). For example, ONTAK is an FDA approved drug used as a cytotoxic therapy for certain T cell malignancies. It consists of a protein conjugate of IL-2 with diphtheria toxin. IL-2 binds to cells bearing the high affinity receptor for IL-2 (CD25), permitting targeted entry of the diphtheria toxin into CD25 positive cells. ONTAK is also capable of inhibiting regulatory T cells.

Amino acid sequences of FDA approved protein/peptide drugs that possess Treg inhibition capabilities as well as the amino acid sequences of drugs undergoing clinical development may be derivatized, for example, bearing modifications other than insertion, deletion, or substitution of amino acid residues, thus resulting in a variation of the original product (a variant). These modifications can be covalent in nature, and include for example, chemical bonding with lipids, other organic moieties, inorganic moieties, and polymers. For additional reviews, please see Foss (2006) Semin Oncol. 33(1 Suppl 3):S11-6; Bayes et al., (2006) Methods Find Exp Clin Pharmacol. 28(4):233-77; Morse et al., (2005) Curr Opin Mol. Ther. 7(6):588-97; Eklund et al., (2005) Expert Rev Anticancer Ther. 5(1):33-8; Sandrini (2005) Clin Transplant 19(6):705-10; Smith et al., (2003) Pediatr Clin North Am. 50(6): 1283-300.

Inhibition of RNA can effectively inhibit expression of a gene from which the RNA is transcribed, for example can inhibit genes with immunosuppressive or immunostimulatory roles. Inhibitors are selected from the group comprising: siRNA, interfering RNA or RNAi; dsRNA; RNA Polymerase III transcribed DNAs; ribozymes; and antisense nucleic acid, which may be RNA, DNA, or artificial nucleic acid. Also within the scope of the present invention are oligonucleotide sequences that include antisense oligonucleotides and ribozymes that function to bind to, degrade and/or inhibit the translation of an mRNA encoding a Treg cell surface marker, such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1, and the like.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a Treg cell surface marker polypeptide can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59).

siRNA comprises a double stranded structure typically containing 15 to 50 base pairs and preferably 21 to 25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The inhibitor may be polymerized in vitro, recombinant RNA, contain chimeric sequences, or derivatives of these groups. The inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded. (see for example Bass (2001) Nature, 411, 428 429; Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914).

Ribozymes are enzymatic RNA molecules capable of catalyzing the specific cleavage of RNA. The mechanism of ribozyme action involves sequence specific hybridization of the ribozyme molecule to complementary target RNA encoding the Treg cell surface marker, followed by endonucleolytic cleavage. Engineered hammerhead motif ribozyme molecules that specifically and efficiently catalyze endonucleolytic cleavage of mRNA sequences encoding a Treg cell surface marker, such as CD25, CD4, CD28, CD38, CD62L (selectin), OX-40 ligand (OX-40L), CTLA4, CCR4, CCR8, FOXP3, LAG3, CD103, NRP-1, glucocorticoid-induced TNF receptor (GITR), galectin-1, TNFR2, or TGF-βR1, and the like, are also within the scope of the present invention. Scanning the target molecule for ribozyme cleavage sites that include the following sequences, GUA, GUU, and GUC initially identifies specific ribozyme cleavage sites within any potential RNA target. Once identified, short RNA sequences of between about 15 and 20 ribonucleotides corresponding to the region of the target gene containing the cleavage site can be evaluated for predicted structural features such as secondary structure that may render the oligonucleotide sequence unsuitable. The suitability of candidate targets can also be evaluated by testing their accessibility to hybridization with complementary oligonucleotides using, e.g., ribonuclease protection assays.

Both the antisense oligonucleotides and ribozymes of the present invention can be prepared by known methods. These include techniques for chemical synthesis such as, e.g., by solid phase phosphoamite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters.

Various modifications to the oligonucleotides of the present invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Aptamers nucleic acid sequences are readily made that bind to a wide variety of target molecules. The aptamer nucleic acid sequences of the invention can be comprised entirely of RNA or partially of RNA, or entirely or partially of DNA and/or other nucleotide analogs. Aptamers are typically developed to bind particular ligands by employing known in vivo or in vitro (most typically, in vitro) selection techniques known as SELEX (Systematic Evolution of Ligands by Exponential Enrichment). Methods of making aptamers are described in, for example, Ellington and Szostak (1990) Nature 346:818, Tuerk and Gold (1990) Science 249:505, U.S. Pat. No. 5,582,981; PCT Publication No. WO 00/20040; U.S. Pat. No. 5,270,163; Lorsch and Szostak (1994) Biochem. 33:973; Mannironi et al., (1997) Biochem. 36:9726; Blind (1999) Proc. Nat'l. Acad. Sci. USA 96:3606-3610; Huizenga and Szostak (1995) Biochem. 34:656-665; PCT Publication Nos. WO 99/54506, WO 99/27133, and WO 97/42317; and U.S. Pat. No. 5,756,291.

Generally, in their most basic form, in vitro selection techniques for identifying RNA aptamers involve first preparing a large pool of DNA molecules of the desired length that contain at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. The oligonucleotide pool is amplified using standard PCR techniques. The DNA pool is then transcribed in vitro. The RNA transcripts are then subjected to affinity chromatography. The transcripts are most typically passed through a column or contacted with magnetic beads or the like on which the target ligand has been immobilized. RNA molecules in the pool, which bind to the ligand, are retained on the column or bead, while nonbinding sequences are washed away. The RNA molecules, which bind the ligand, are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules that are capable of acting as aptamers for the target ligand.

One can generally choose a suitable ligand without reference to whether an aptamer is yet available. In most cases, an aptamer can be obtained which binds the small, organic molecule of choice by someone of ordinary skill in the art. The unique nature of the in vitro selection process allows for the isolation of a suitable aptamer that binds a desired ligand despite a complete dearth of prior knowledge as to what type of structure might bind the desired ligand.

The association constant for the aptamer and associated ligand is, for example, such that the ligand functions to bind to the aptamer and have the desired effect at the concentration of ligand obtained upon administration of the ligand. For in vivo use, for example, the association constant should be such that binding occurs below the concentration of ligand that can be achieved in the serum or other tissue (such as ocular vitreous fluid). For example, the required ligand concentration for in vivo use is also below that which could have undesired effects on the organism.

The aptamer nucleic acid sequences, in addition to including RNA, DNA and mixed compositions, may be modified. For example, certain modified nucleotides can confer improved characteristic on high-affinity nucleic acid ligands containing them, such as improved in vivo stability or improved delivery characteristics. Examples of such modifications include chemical substitutions at the ribose and/or phosphate and/or base positions. SELEX-identified nucleic acid ligands containing modified nucleotides are described in U.S. Pat. No. 5,660,985, entitled “High Affinity Nucleic Acid Ligands Containing Modified Nucleotides,” that describes oligonucleotides containing nucleotide derivatives chemically modified at the 5- and 2′-positions of pyrimidines. U.S. Pat. No. 5,637,459, supra, describes highly specific nucleic acid ligands containing one or more nucleotides modified with 2′-amino (2′—NH.sub.2), 2′-fluoro (2′-F), and/or 2′-O-methyl (2′-OMe). U.S. application Ser. No. 08/264,029, filed Jun. 22, 1994, entitled “Novel Method of Preparation of Known and Novel 2′ Modified Nucleosides by Intramolecular Nucleophilic Displacement,” describes oligonucleotides containing various 2′-modified pyrimidines.

The aptamer nucleic acid sequences of the invention further may be combined with other selected oligonucleotides and/or non-oligonucleotide functional units as described in U.S. Pat. No. 5,637,459, entitled “Systematic Evolution of Ligands by Exponential Enrichment: Chimeric SELEX,” and U.S. Pat. No. 5,683,867, entitled “Systematic Evolution of Ligands by Exponential Enrichment: Blended SELEX,” respectively.

A Treg agent can also be a small molecule that binds to a Treg cell surface marker and disrupts its function. Small molecules are a diverse group of synthetic and natural substances generally having low molecular weights. They are isolated from natural sources (for example, plants, fungi, microbes and the like), are obtained commercially and/or available as libraries or collections, or synthesized. Candidate Treg agent small molecules can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described below (Werner et al., (2006) Brief Funct. Genomic Proteomic 5(1):32-6).

Diversity libraries, such as random or combinatorial peptide or non-peptide libraries can be screened for small molecules and compounds that specifically bind to Treg cell surface markers. Many libraries are known in the art that can be used such as, e.g., chemically synthesized libraries, recombinant (e.g., phage display) libraries, and in vitro translation-based libraries.

Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Candidate Treg agents or inhibitors can be screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. Libraries are also meant to include for example but are not limited to peptide-on-plasmid libraries, polysome libraries, aptamer libraries, synthetic peptide libraries, synthetic small molecule libraries and chemical libraries. The libraries can also comprise cyclic carbon or heterocyclic structure and/or aromatic or polyaromatic structures substituted with one or more of the above-identified functional groups.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383.

Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/18318.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318.

According to the method of the invention, a Treg agent modulates a T regulatory cell via either decreasing the activity or function of a Treg after the Treg agent is administered to a subject; or a Treg agent that is attached to a toxic moiety can kill or ablate T regulatory cells. The administration of a Treg agent or derivatives thereof can block the action of its target (for example a Treg cell surface marker). Thus, a Treg agent can decrease the suppression of immune system activation and can decrease the prevention of self-reactivity. Such a decrease can be measured via techniques established in the art. For example, see Dannull et al., (2005) J Clin Invest 115(12):3623-33; and Tsaknaridis, et al., (2003) J Neurosci Res 74: 296-308. Non-limiting examples of assays used for the detection of T cell responses include delayed-type hypersensitivity responses; in vitro T cell proliferation responses (e.g., measured by incorporation of radioactive nucleotides); library screens; expression arrays; T cell cytokine responses (e.g., measured by ELISA or other related immuno-assays or RT-PCR for specific cytokine mRNA); as well as any other assay established in the art for measuring a B cell and/or T cell immune response in a subject. Methods for detecting an immune response can include, but are not limited to, antibody detection assays such as, for example, EIA (enzyme immunoassay); ELISA (enzyme linked immunosorbent assay); agglutination reactions; precipitation/flocculation reactions, immunoblots (Western blot; dot/slot blot); (RIA) radioimmunoassays; immunodiffusion assays; histochemical assays; immunofluorescence assays (FACS); chemiluminescence assays, library screens, expression arrays, etc.

Methods of Treatment

The invention provides a method of converting passive immunotherapy with antibodies into an active immunization protocol via inhibition of T regulatory cell function coupled with increasing the number of immune complexes comprising one or more target antigens in a subject. The method provides a synergistic effect with respect to effector cell functions as compared to the administration of immune complexes alone, antibodies alone, or Treg agents alone. The methods of converting a passive immunization into active immunity against a target can be the basis for a method of treating a disease or disorder. The particular disease or disorder treated is based on the target antigen(s) of the antibodies administered to the subject, i.e., the antigen that is bound by the administered antibodies resulting in increased immune complex formation.

In one embodiment, the methods of the invention can be methods for treating cancers, including, for example: B cell lymphoma, colon cancer, lung cancer, renal cancer, bladder cancer, T cell lymphoma, myeloma, leukemia, chronic myeloid leukemia, acute myeloid leukemia, chronic lymphocytic leukemia, acute lymphocytic leukemia, hematopoietic neoplasias, thymoma, lymphoma, sarcoma, lung cancer, liver cancer, non-Hodgkins lymphoma, Hodgkins lymphoma, uterine cancer, renal cell carcinoma, hepatoma, adenocarcinoma, breast cancer, pancreatic cancer, liver cancer, prostate cancer, head and neck carcinoma, thyroid carcinoma, soft tissue sarcoma, ovarian cancer, primary or metastatic melanoma, squamous cell carcinoma, basal cell carcinoma, brain cancer, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, astrocytoma, medulloblastoma, craniopharyngioma, ependymoma, pinealoma, hemangioblastoma, acoustic neuroma, oligodendroglioma, meningioma, retinoblastoma, leukemia, melanoma, neuroblastoma, small cell lung carcinoma, bladder carcinoma, lymphoma, multiple myeloma, and medullary carcinoma.

The methods of the invention can also be methods of therapy for diseases caused by pathogenic infections. Such infections can be generated by bacteria, fungi, protozoa, viruses, parasites, and the like. Non-limiting examples of diseases caused by viral infections include AIDS, AIDS Related Complex, Chickenpox (Varicella), Common cold, asthma, viral bronchitis, Cytomegalovirus Infection, Colorado tick fever, Dengue fever, Ebola haemorrhagic fever, Epidemic parotitis, Hand, foot and mouth disease, Hepatitis, Herpes simplex, Herpes zoster, HPV, Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease, and Yellow fever.

Diseases caused by bacterial infections include, but are not limited to, Anthrax, bacterial adult respiratory distress syndrome, Bacterial Meningitis, Brucellosis, Campylobacteriosis, Cat Scratch Disease, bronchitis, Cholera, chronic obstructive pulmonary disease (COPD), Diphtheria, Epidemic Typhus, Gonorrhea, Impetigo, Legionellosis, Leprosy (Hansen's Disease), Leptospirosis, Listeriosis, Lyme Disease, Melioidosis, MRSA infection, mycobacterial infection, meningitis, Nocardiosis, nephritis, glomerulonephritis, periodontal disease, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, septic shock, haemodynamic shock, sepsis syndrome, Tetanus, Trachoma, Tuberculosis, Tularemia, Typhoid Fever, and Typhus.

Non-limiting examples of parasitic infections, which can be also be caused by some parasitic protozoans, that can be subject to the methods of the invention include African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, and Trypanosomiasis.

Fungal infectious diseases include, but are not limited to Aspergillosis, Blastomycosis, Candidiasis, Coccidioidomycosis, Cryptococcosis, Histoplasmosis, Sepsis, and Tinea pedis.

Combined therapy with both a chemotherapy drug and an anti-tumor antibody concurrently or soon after a Treg agent has been administered is a method that additively (with respect to the chemotherapy drug and the co-administration of the anti-tumor antibody (or ICs) and the Treg agent) induces the direct killing of a tumor. In several embodiments, the methods of the invention comprise co-administering an agent that inhibits Tregs with an anti-tumor antibody in order to invoke the adaptive immune system and treat diseases and conditions, including those associated with cancer.

In certain embodiments, the invention provides for methods of treating or reducing cancer in a subject by inhibiting/depleting T regulatory cells and by increasing immune complex number (which comprise cancer antigens) in the subject in order to promote the efficacy of chemotherapy drugs. The invention also provides for methods of preventing the progression of cancer in a subject to promote the efficacy of chemotherapy drugs. The method comprises administering an effective amount of an agent to a subject, wherein the agent decreases the activity or function of a regulatory T cell (Treg), in addition to an effective amount of an anti-tumor antibody (or immune complexes comprising antibodies that target tumor antigens and the antigens themselves), which results in treating or reducing cancer in the subject. This treatment results in the conversion of passive immunotherapy into active immunization as discussed above. In addition, the method can further comprise the administering of a chemotherapy drug. If a chemotherapy drug is used, the method may or may not comprise the administering of an anti-tumor antibody.

Cytotoxic drugs (for example, chemotherapy drugs) that interfere with critical cellular processes including DNA, RNA, and protein synthesis, can be conjugated to antibodies and ligands and used for in vivo therapy or be used without the modifications just described. Some non-limiting examples of conventional chemotherapy drugs include: aminoglutethimide, amsacrine, asparaginase, bcg, anastrozole, bleomycin, buserelin, bicalutamide, busulfan, capecitabine, carboplatin, camptothecin, chlorambucil, cisplatin, carmustine, cladribine, colchicine, cyclophosphamide, cytarabine, dacarbazine, cyproterone, clodronate, daunorubicin, diethylstilbestrol, docetaxel, dactinomycin, doxorubicin, dienestrol, etoposide, exemestane, filgrastim, fluorouracil, fludarabine, fludrocortisone, epirubicin, estradiol, gemcitabine, genistein, estramustine, fluoxymesterone, flutamide, goserelin, leuprolide, hydroxyurea, idarubicin, levamisole, imatinib, lomustine, ifosfamide, megestrol, melphalan, interferon, irinotecan, letrozole, leucovorin, ironotecan, mitoxantrone, nilutamide, medroxyprogesterone, mechlorethamine, mercaptopurine, mitotane, nocodazole, octreotide, methotrexate, mitomycin, paclitaxel, oxaliplatin, temozolomide, pentostatin, plicamycin, suramin, tamoxifen, porfimer, mesna, pamidronate, streptozocin, teniposide, procarbazine, titanocene dichloride, raltitrexed, rituximab, testosterone, thioguanine, vincristine, vindesine, thiotepa, topotecan, tretinoin, vinblastine, trastuzumab, and vinorelbine.

In one embodiment, the chemotherapy drug is an alkylating agent, a nitrosourea, an anti-metabolite, a topoisomerase inhibitor, a mitotic inhibitor, an anthracycline, a corticosteroid hormone, a sex hormone, or a targeted anti-tumor compound.

A targeted anti-tumor compound is a drug designed to attack cancer cells more specifically than standard chemotherapy drugs can. Most of these compounds attack cells that harbor mutations of certain genes, or cells that overexpress copies of these genes. In one embodiment, the anti-tumor compound can be imatinib (Gleevec), gefitinib (Iressa), erlotinib (Tarceva), rituximab (Rituxan), or bevacizumab (Avastin).

An alkylating agent works directly on DNA to prevent the cancer cell from propagating. These agents are not specific to any particular phase of the cell cycle. In one embodiment, alkylating agents can be selected from busulfan, cisplatin, carboplatin, chlorambucil, cyclophosphamide, ifosfamide, dacarbazine (DTIC), mechlorethamine (nitrogen mustard), melphalan, and temozolomide.

An antimetabolite makes up the class of drugs that interfere with DNA and RNA synthesis. These agents work during the S phase of the cell cycle and are commonly used to treat leukemias, tumors of the breast, ovary, and the gastrointestinal tract, as well as other cancers. In one embodiment, an antimetabolite can be 5-fluorouracil, capecitabine, 6-mercaptopurine, methotrexate, gemcitabine, cytarabine (ara-C), fludarabine, or pemetrexed.

Topoisomerase inhibitors are drugs that interfere with the topoisomerase enzymes that are important in DNA replication. Some examples of topoisomerase I inhibitors include topotecan and irinotecan while some representative examples of topoisomerase II inhibitors include etoposide (VP-16) and teniposide.

Anthracyclines are chemotherapy drugs that also interfere with enzymes involved in DNA replication. These agents work in all phases of the cell cycle and thus, are widely used as a treatment for a variety of cancers. In one embodiment, an anthracycline used with respect to the invention can be daunorubicin, doxorubicin (Adriamycin), epirubicin, idarubicin, or mitoxantrone.

According to the invention, after co-administration of an antibody directed at a pathogenic antigen and a Treg agent, combined therapy can also encompass administering an antibiotic an anti-fungal drug, an anti-viral drug, an anti-parasitic drug, an anti-protozoal drug, or a combination thereof. This method additively (co-administration plus the anti-pathogenic agent) induces the elimination of a pathogenic infection (such as those described above). The invention provides a method of converting passive immunotherapy with antibodies into an active immunization protocol via elimination/inhibition of T regulatory cell function coupled with an immune complex-FcR mediated mechanism (such as activation of APC), wherein the method comprises co-administering an agent that inhibits Tregs with an antibody directed against a pathogenic antigen in order to invoke the immune system and treat diseases and conditions associated with pathogenic infections.

The efficacy of antibiotics, antifungal agents, antiviral agents, anti-parasite drugs, or anti-protozoal compounds can be enhanced according to the methods of the invention. An antibiotic refers to any compound known to one of ordinary skill in the art that will inhibit the growth of, or kill, bacteria. Useful, non-limiting examples of an antibiotic include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); vancomycins; bacitracins; macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Perfloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. See also Physician's Desk Reference, 59^{th} edition, (2005), Thomson P D R, Montvale N.J.; Gennaro et al., Eds. Remington's The Science and Practice of Pharmacy, 20^{th} edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J. Such antibiotics can be obtained commercially, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

An anti-fungal agent refers to any compound known to one of ordinary skill in the art that will inhibit the growth of, or kill, fungi. Non-limiting examples include imidazoles (such as griseofulvin, miconazole, terbinafine, fluconazole, ketoconazole, voriconazole, and itraconizole); polyenes (such as amphotericin B and nystatin); Flucytosines; and candicidin or any salts or variants thereof. See also Physician's Desk Reference, 59^{th} edition, (2005), Thomson P D R, Montvale N.J.; Gennaro et al., Eds. Remington's The Science and Practice of Pharmacy 20^{th} edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.

An anti-viral drug refers to any compound known to one of ordinary skill in the art that will inhibit action of a virus. Non-limiting examples include interferon alpha, beta or gamma, didanosine, lamivudine, zanamavir, lopanivir, nelfinavir, efavirenz, indinavir, valacyclovir, zidovudine, amantadine, rimantidine, ribavirin, ganciclovir, foscarnet, and acyclovir or any salts or variants thereof. See also Physician's Desk Reference, 59^{th} edition, (2005), Thomson P D R, Montvale N.J.; Gennaro et al., Eds. Remington's The Science and Practice of Pharmacy 20^{th} edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.

An anti-parasitic agent refers to any compound known to one of ordinary skill in the art that will inhibit the growth of, or kill, parasites (such as those previously described). Useful, non-limiting examples of an anti-parasitic agent include chloroquine, mefloquine, quinine, primaquine, atovaquone, sulfasoxine, and pyrimethamine or any salts or variants thereof. See also Physician's Desk Reference, 59^{th} edition, (2005), Thomson P D R, Montvale N.J.; Gennaro et al., Eds. Remington's The Science and Practice of Pharmacy 20^{th} edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.

Anti-protozoal drug refers to any compound known to one of ordinary skill in the art that will inhibit the growth of, or kill, protozoa. Useful, non-limiting examples include metronidazole, diloxanide, iodoquinol, trimethoprim, sufamethoxazole, pentamidine, clindamycin, primaquine, pyrimethamine, and sulfadiazine or any salts or variants thereof. See also Physician's Desk Reference, 59^{th} edition, (2005), Thomson P D R, Montvale N.J.; Gennaro et al., Eds. Remington's The Science and Practice of Pharmacy 20^{th} edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.

The invention also provides for methods of treating or reducing cancer in a subject. The method according to the invention can also be applicable for preventing progression of cancer in a subject. Use of the Treg agents in these two aspects of the invention can promote the efficacy of chemotherapy drugs. Co-administration of a chemotherapy drug and an agent that decreases the activity or function of a regulatory T cell, increases T cell proliferative responses due to the increase of effector T cells. The method comprises administering an effective amount of an agent to a subject, wherein the agent decreases the activity or function of a regulatory T cell (Treg), and administering an effective amount of an anti-tumor antibody (or immune complex), whereby the combination treatment results in treating or reducing cancer in the subject. In another aspect of the invention the combination treatment results in preventing the progression of cancer in the subject. In addition, the method can further comprise administering a chemotherapy drug. If a chemotherapy drug is used, the method may or may not comprise the administering of an anti-tumor antibody.

In one embodiment, an agent that decreases the activity or function of a regulatory T cell and a chemotherapy drug are administered simultaneously. In another embodiment, an agent that decreases the activity or function of a regulatory T cell and a chemotherapy drug are administered sequentially.

Passive immunotherapy is converted into active immunization in the present invention, as discussed above. In one embodiment, the method comprises the use of antibodies directed against Treg cell surface markers in combination with a chemotherapy drug. In other embodiments, Treg cell surface marker antibodies (for example, polyclonal, monoclonal, humanized, and the like) are directed at CD4, CD25, CD28, CTLA4, CCR4, CCR8, LAG3, CD103, NRP-1, or GITR. In further embodiments, FDA-approved drug(s) that possess the ability to inhibit regulatory T cells (for example, via decreasing the function or activity of Tregs drug(s) are used. Yet, in other embodiments, drugs under clinical development that can act as Treg inhibitors are used. Some non-limiting examples of Treg inhibitors include ONTAK, HuMax-Tac, Zenapax, and MDX-010. Treatment with FDA-approved drugs that act as Treg inhibitors or antibodies directed against Treg cell surface markers leads to an inhibition of the suppression of an effective anti-tumor response. Thus, inhibiting Treg function and/or activity leads to more responsive chemotherapy drugs, converting this passive therapy into an active therapy.

In addition, the current invention also provides methods for treating or reducing a pathogenic infection in a subject via using Treg agents. This method can promote the efficacy of subsequent drugs administered, such as antibiotics, antifungal or antiviral agents, as well as anti-parasite and anti-protozoal compounds. The method comprises administering an effective amount of an agent to a subject, wherein the agent decreases the activity or function of a regulatory T cell (Treg), in addition to administering an effective amount of an antibody directed at a pathogenic antigen. The combination treatment results in treating or reducing the pathogenic infection in the subject. In addition, the method further comprises the conversion of passive immunotherapy into active immunization as discussed above. The method can further comprise administering an antibiotic, antifungal agent, antiviral agent, anti-parasite drug, or an anti-protozoal compound. Thus, the present invention provides for co-administration of an anti-pathogen drug (such as an antibiotic, antifungal agent, antiviral agent, anti-parasite drug, an anti-protozoal compound, and the like), an antibody directed to a pathogenic antigen, and an agent that decreases the activity or function of a regulatory T cell, which can result in T cell proliferative responses due to the generation of effector T cells. In some embodiments, co-administration of agents and drugs occurs simultaneously while in other embodiments, the agent and drugs are administered sequentially.

Passive immunotherapy is converted into active immunization in the present invention, as previously discussed. In one embodiment, the use of antibodies directed against Treg cell surface markers in combination with a chemotherapy drug decreases the function or activity of Tregs. In other embodiments, Treg cell surface marker antibodies (for example, polyclonal, monoclonal, humanized, and the like) are directed at CD4, CD25, CD28, CTLA4, CCR4, CCR8, LAG3, CD103, NRP-1, or GITR. In further embodiments, FDA-approved drug(s) that possess the ability to inhibit regulatory T cells (for example, via decreasing the function or activity of Tregs drug(s) are used. Yet, in other embodiments, drugs under clinical development that can act as Treg inhibitors are used. Non-limiting examples of Treg inhibitors have been described above. Inhibiting Treg function and/or activity leads to more responsive antibodies directed at pathogenic antigens, thus converting this passive therapy into an active therapy.

According to the methods of the invention wherein an infection is treated or reduced, any pathogenic entity can cause the infection. Non-limiting examples of pathogenic entities have been previously described.

According to the methods of the invention wherein an infection is treated or reduced, the efficacy of antibiotics, antifungal agents, antiviral agents, anti-parasite drugs, or an anti-protozoal compounds can be enhanced. A subject in need of treatment (for example those previously described, such as an animal or human) can be one afflicted with the infections or disorders caused by the various pathogens described above. Such a subject at risk could be a candidate for treatment with a Treg agent co-administered with an antibody directed at a pathogenic antigen. Additionally, antibiotics, antifungal agents, antiviral agents, anti-parasite drugs, or an anti-protozoal compounds can be administered to the subject. Such a treatment could inhibit the development or onset of a pathogen-associated disorder/condition or prevent the recurrence, onset, or development of one or more symptoms of a pathogen-associated disorder/condition.

The subject in need can be administered a Treg agent as described above in combination with an antibody directed at a pathogenic antigen. They can be administered alone or in combination with a third therapeutic, e.g., such as an antibiotic, antifungal agent, antiviral agent, anti-parasite drug, or an anti-protozoal compound, in order to treat or reduce a pathogenic infection. The third group of therapeutics can be co-administered with the Treg agent and antibody, either sequentially or simultaneously.

The reagent that reduces or inhibits immunosuppressive activity of regulatory T cells in a subject can be administered to the subject at least zero, one, two, three, four, five, six, seven, eight, nine or ten days before a reagent that acts to elicit an immune response (e.g., to treat cancer or an infection) is administered to the subject. In one embodiment, the Treg agent and antibody directed against a tumor antigen are administered simultaneously. In other embodiments, the Treg agent and anti-tumor antibody is administered sequentially. According to methods of the invention, the T cell response is promoted by prior removal or inhibition of functional regulatory T cells. For example, ONTAK binds and kills CD25-bearing cells and ONTAK then would be expected to kill both regulatory T cells and recently activated conventional T cells (which transiently express CD25 for a few days). Since the half-life of ONTAK is a few minutes, it may be administered simultaneously with an anti-tumor antibody. With other regulatory T cell inhibiting agents (e.g. GITR antibodies or CTLA4 antibodies), cross-reactivity would not be expected with conventional T cells since those antibodies are directed at cell surface markers specific for Tregs that are not present on conventional T cells.

In some embodiments of the invention, the Treg agent (such as Treg cell surface marker antibodies directed to CD4, CD25, CD28, CTLA4, CCR4, CCR8, LAG3, CD103, NRP-1, or GITR; and FDA-approved drugs capable of inhibiting Tregs such as ONTAK, HuMax-Tac, Zenapax, and MDX-010) is administered only once to the subject. In other embodiments, the reagent is administered more than once to the subject, at an interval deemed to have a therapeutic effect. The skilled physician can determine the therapeutic interval. In further embodiments, the reagent can be administered so that a specified amount of the reagent is maintained in the subject for a given period of time. Yet, in other embodiments of the invention, the reagent is administered such that it is present in the subject only transiently.

When the Treg inhibitor of the current invention is a fusion protein, the amount of fusion protein administered can be in a range from about 5 μg/kg to about 40 μg/kg. For example, ONTAK can be administered at a dose of 5 μg/kg of body weight per day. ONTAK can be given intravenously for three consecutive days every other week for up to eight weeks. The dose of 5 μg/kg is one-half to one-quarter the current dose of ONTAK approved by the FDA for treatment of patients with cutaneous T-cell lymphoma (CTCL) malignant cells.

When the Treg inhibitor is an antibody directed at a Treg cell surface marker, the amount of the antibody administered can be in a range from about 1 mg/kg to about 5 mg/kg. For example, Zenapax can be administered at a dose of 1 mg/kg of body weight per day while MDX-010 can be administered at a dose of 3 mg/kg.

When the anti-tumor antibody is an antibody directed at a cancer antigen (such as HER2, in addition to those listed above), the amount of the antibody administered can be in a range from about 1 mg/kg to 10 mg/kg. For example, the recommended initial loading dose of an anti-HER2 antibody (such as Herceptin or trastuzumab) is 4 mg/kg of body weight administered as a 90-minute infusion. The recommended weekly maintenance dose is 2 mg/kg and can be administered as a 30-minute infusion.

The skilled physician via the published literature or clinical trials can determine the efficacy and toxicity of a chemotherapy drug. The skilled physician can also determine a therapeutic dose of a chemotherapy drug that inhibits and/or treats cancer in a subject in addition that prevents and/or reduces the progression of cancer in a subject. For example, Cytoxan can be used at doses around 300 mg/m² (Berd and Mastrangelo (1988) Cancer Res. 48(6):1671-5; Berd and Mastrangelo (1987) Cancer Res. 47(12):3317-21). For further review please see Gennaro et al., Eds. Remington's The Science and Practice of Pharmacy 20^{th} edition, (2000), Lippincott Williams and Wilkins, Baltimore Md., Chapter 86; Braunwald et al., Eds. Harrison's Principles of Internal Medicine, 15^{th} edition, (2001), McGraw Hill, NY, Chapter 84; Berkow et al., Eds. The Merck Manual of Diagnosis and Therapy, (1992), Merck Research Laboratories, Rahway N.J., pages 1277-81; Petrulio et al., (2006) Expert Opin Biol Ther. 6(7):671-84; and Ehrke et al., (1989) Semin Oncol. 16(3):230-53.

If the Treg agent is to be administered to a subject, it will be in the form of a pharmaceutically acceptable composition or formulation as described below, wherein the composition or formulation is free of toxicity, which satisfies FDA requirements (see Remington: The Science and Practice of Pharmacy 20^{th} ed., Lippincott Williams & Wilkins, 2000; U.S. Pat. No. 6,030,604). Such a Treg agent composition, comprising compounds or pharmaceutically acceptable salts, can be administered to a subject afflicted with cancer or a pathogenic infection. Administration can occur alone or with other therapeutically effective composition(s) (e.g., antibiotics, chemotherapy drugs, and the like) either simultaneously or at different times.

Formulations can include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form, will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) each containing a predetermined amount of a compound of the present invention as an active ingredient. A compound of the present invention may also be administered as a bolus, electuary, or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used, include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The Treg agent composition can optionally comprise a suitable amount of a physiologically acceptable excipient. Non-limiting examples of physiologically acceptable excipients can be liquids, such as water and oils, including those of petroleum, animal, vegetable, or synthetic origin, such as peanut oil, soybean oil, mineral oil, sesame oil and the like; saline; gum acacia; gelatin; starch paste; talc; keratin; colloidal silica; urea and the like. In addition, auxiliary, stabilizing, thickening, lubricating, and coloring agents can be used. For example, the Treg agent composition and physiologically acceptable excipient are sterile when administered to a subject (such as an animal; for example a human). The physiologically acceptable excipient should be stable under the conditions of manufacture and storage and should be preserved against the contaminating action of microorganisms.

Water is a useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The Treg agent composition can be administered to the subject by any effective route, for example, orally, by infusion or bolus injection, by absorption through epithelial or mucocutaneous linings (e.g., oral, rectal, vaginal, and intestinal mucosa, etc.), intradermal, intramuscular, intraperitoneal, intravenous, subcutaneous, infusion, intranasal, epidural, oral, sublingual, intracerebral, intravaginal, transdermal, rectal, by inhalation, or topical, particularly to the ears, nose, eyes, or skin.

The Treg agent composition can be delivered in a vesicle, in particular a liposome (see Langer, (1990) Science 249:1527-1533; and Treat et al., (1989) Liposomes in the Therapy of Infectious Disease and Cancer 317-327 and 353-365). The Treg agent composition also can be delivered in a controlled-release system or sustained-release system (see, e.g. Goodson, (1984) in Medical Applications of Controlled Release, vol. 2, pp. 115-138). Other controlled or sustained-release systems discussed in the review by Langer, (1990) Science 249:1527-1533 can be used. In one embodiment, a pump can be used (Langer, (1990) Science 249:1527-1533; Sefton, (1987) CRC Crit. Ref Biomed. Eng. 14:201; Buchwald et al., (1980) Surgery 88:507; and Saudek et al., (1989) N. Engl. J Med. 321:574). In another embodiment, polymeric materials can be used (see Controlled Drug Bioavailability, Drug Product Design and Performance (Smolen and Ball eds., 1984); Ranger and Peppas, (1983) J. Macromol. Sci. Rev. Macromol. Chem. 2:61; Levy et al., (1935) Science 228:190; During et al., (1989) Ann. Neural. 25:351; and Howard et al., (1989) J. Neurosurg. 71:105).

### EXAMPLES OF THE INVENTION

A number of Examples are provided below to facilitate a more complete understanding of the present invention. However, the scope of the invention is not limited to specific embodiments disclosed in these Examples, which are for purposes of illustration only.

### Example 1

### Experimental Design

FVB mice were immunized with HER-2 containing immune complexes (ICs which contain both HER-2 protein and rabbit polyclonal anti-HER-2 IgGs) either with or without prior regulatory T cell inhibition with ONTAK:

| Mice | Days −17/−15 | Days −14/−7 | Day 0 |
|---|---|---|---|
| Group 1 | — | — | FVB HER-2 tumor challenge |
| | | | (NT2.4 cells) |
| Group 2 | ONTAK i.p. | — | FVB HER-2 tumor challenge |
| | | | (NT2.4 cells) |
| Group 3 | — | HER-2 ICs i.v | FVB HER-2 tumor challenge |
| | | | (NT2.4 cells) |
| Group 4 | ONTAK i.p. | HER-2 ICs i.v | FVB HER-2 tumor challenge |
| | | | (NT2.4 cells) |

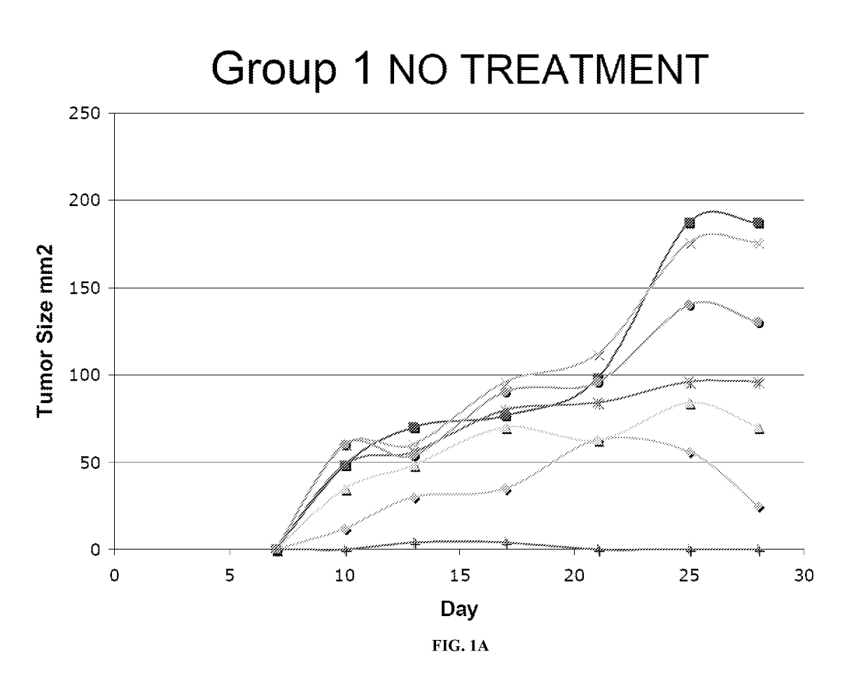

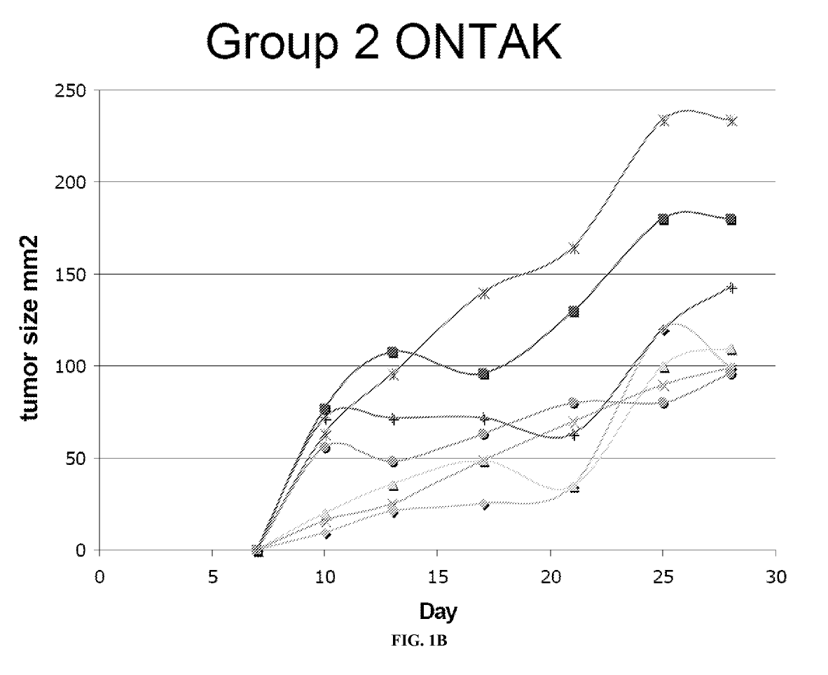

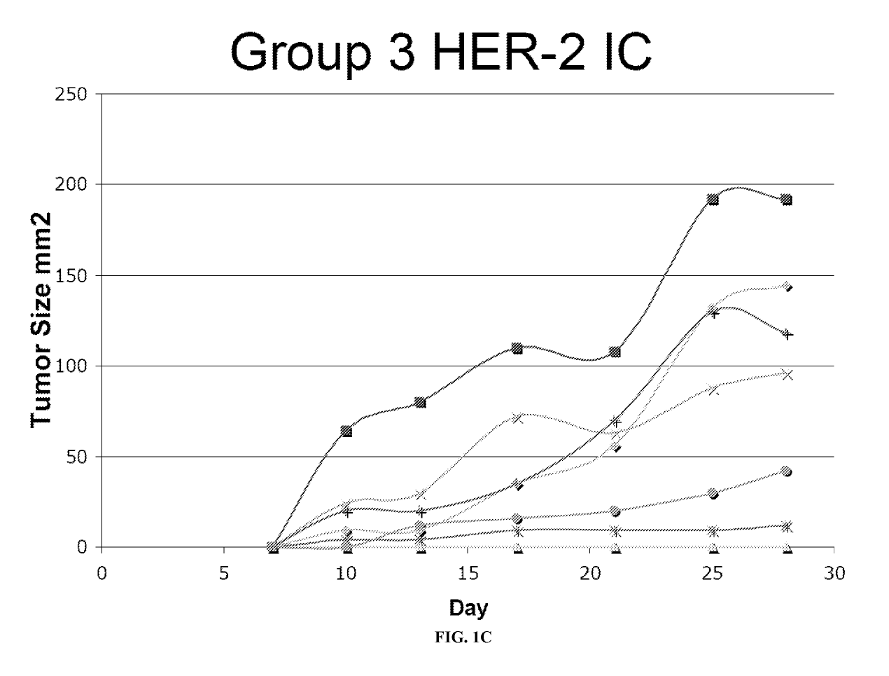

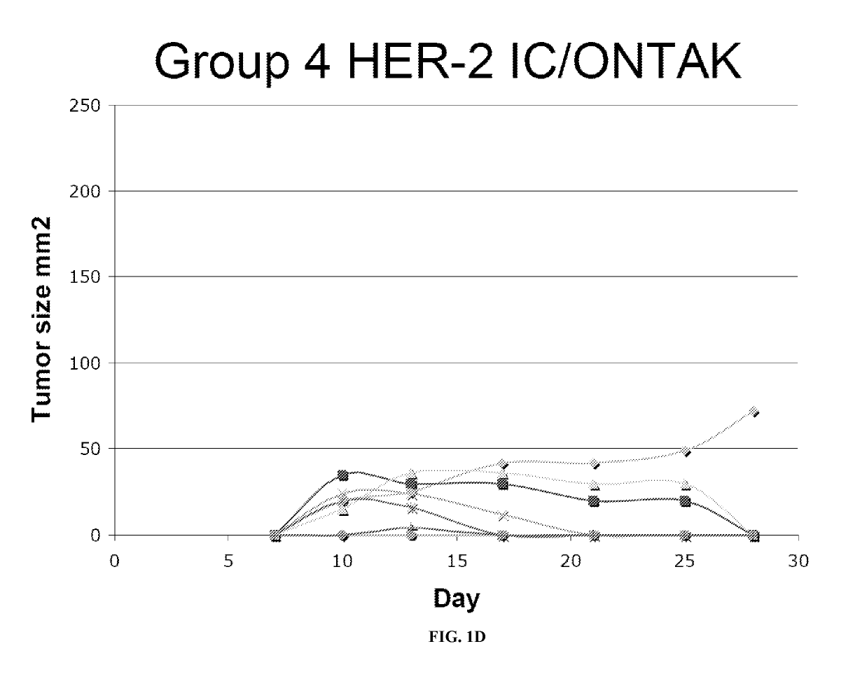

Significant inhibition of tumor growth was seen in Group 4, while there was limited or no protection in Groups 2 or 3 (FIG. 1). Group 1 served as the control.

Therapeutic responses correlated with the induction of HER-2 specific CD4 and CD8 responses, but not humoral antibody responses, suggesting that the protective mechanism was mediated by the induction of a tumor-specific T cell response. Thus, tumor protection and T cell responses in a HER-2 tumor antigen model system showed that inhibition of regulatory T cells has the ability to enhance the potency of vaccines based on an anti-tumor-antibody platform.

### Example 2

To demonstrate that protection was due to an induced T cell response, splenic populations were obtained from immunized mice. Splenocytes were stimulated with either whole Her-2 protein for assessment of CD4 HER-2 specific T cell responses or instead stimulated with a Class I-restricted HER-2 peptide to assess CD8 Her-2 specific responses. After overnight incubation with antigen, T cells were assessed flow cytometrically for IFN-γ production, a cytokine produced by effector CD8 and Th1-type CD4 cells.

HER-2 Specific CD8 Responses:

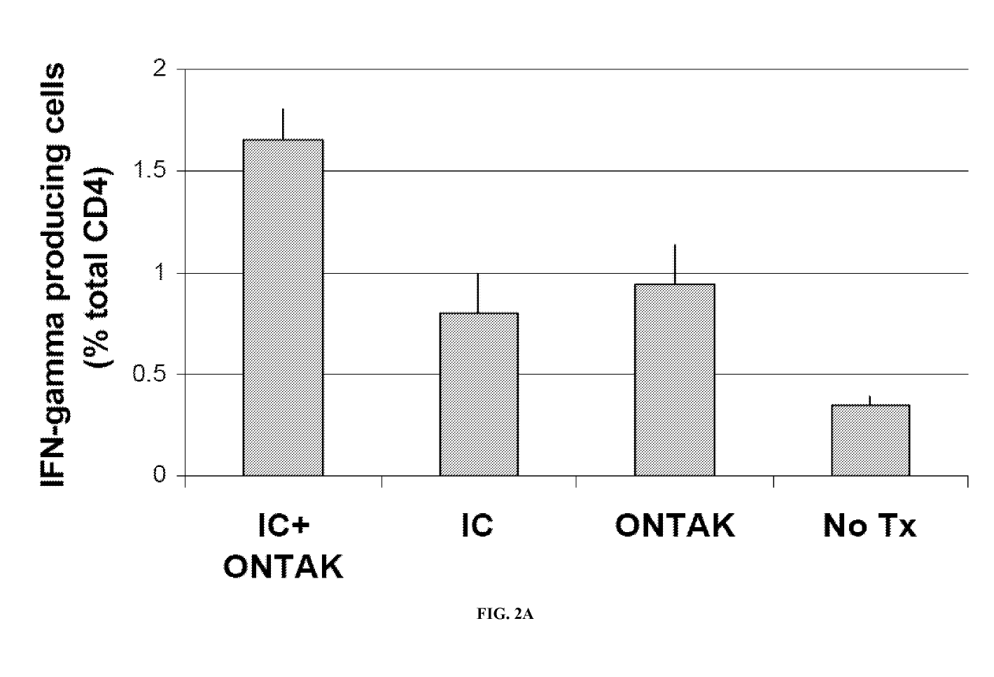

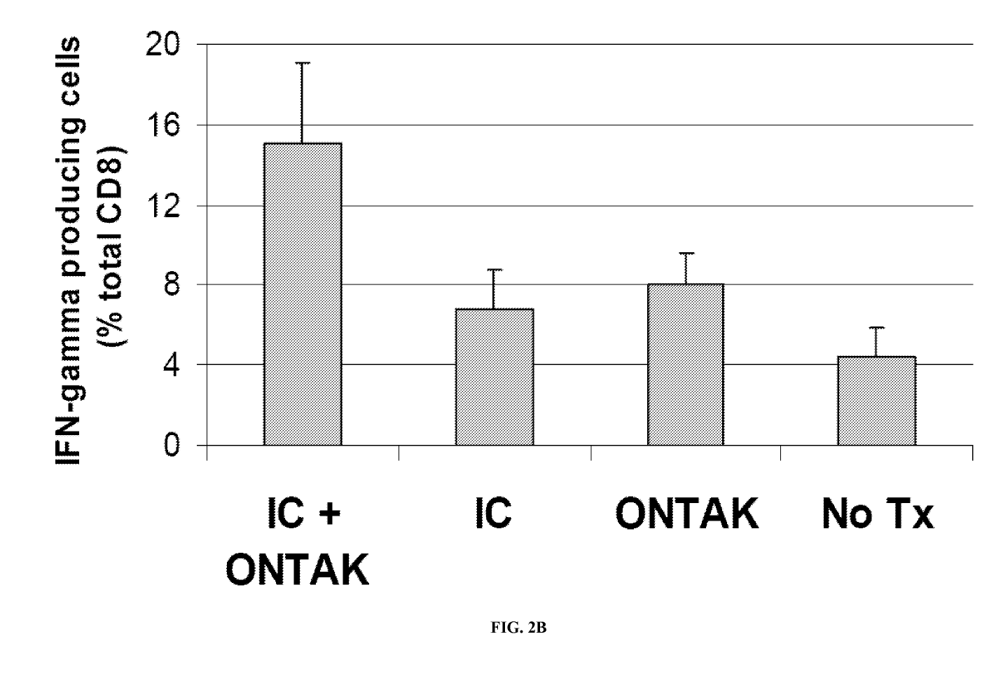

Splenocytes from immunized mice were stimulated with and MHC-I restricted HER-2 peptide overnight and stained for intracellular production of IFN-γ. CD8 cells from mice treated with either ONTAK alone or HER-2 ICs alone demonstrated marginally enhanced IFN-γ production but this did not reach statistical significance (FIG. 2A; Group 1 vs. 2, p=0.2, Group 1 vs. 3, p=0.08). Mice immunized with both HER-2 ICs and ONTAK induced significantly enhanced IFN-γ producing CD8 responses (FIG. 2A; Group 4 vs. 1, p=0.008).

HER-2 Specific CD4 Responses:

Splenocytes from immunized mice were stimulated with HER-2 protein overnight and stained for intracellular production of IFN-γ. CD4 cells from mice treated with both ONTAK and HER-2 ICs demonstrated enhanced IFN-γ production compared with unimmunized mice or mice immunized with either ICs or ONTAK alone (FIG. 2B; Group 4 vs. 1, p=0.00005; group 4 vs. 2, p=0.009; Group 4 vs. group 3, p=0.03).

### Example 3

Inhibition of regulatory T cells augments vaccine induced effector T cell responses. Antibody:antigen containing immune complexes greatly augment antigen presentation and expansion of antigen-specific CD4 and CD8 cells. Combining the administration of anti-tumor antibodies (or tumor antigen immune complexes) with the inhibition of T regulatory cells can be shown in mouse models to enhance anti-tumor immunity, wherein regulatory T cell inhibition prior to administration of anti-tumor antibodies can be employed.

Vaccination of mice with immune complex loaded dendritic cells can induce tumor immunity (Rafiq et al., (2002) J Clin Investig, 110(1):71-9). However, direct immunization of mice with immune complexes fails to induce tumor responses despite triggering impressive expansion of antigen specific T cells. Lack of induction of effector T cell immunity may be due to the coincident induction of regulatory T cell responses.

In the B16 melanoma model, the antibody TA99 recognizes the melanoma antigen TRP-1 and prevents tumor development in a manner dependent on Fc receptor mediated effector responses (Clynes et al., 2000 Nature Medicine 6(4):443-6; Clynes et al., (1998) Proc Natl Acad Sci USA. 95(2):652-6). This anti-tumor antibody fails in treatment models in which antibody therapy is delayed until 7 days after tumor cells are injected.

EXPERIMENTAL METHODS: This section describes and includes the types of cells/tissue (rat, human, etc.) to be used, in vivo/in vitro assays, and endpoints to be evaluated.

MOUSE MODELS: First, two mouse models of tumor immunity will be tested: B16-OVA and B16. B16-OVA is a murine melanoma model that expresses ovalbumin. Immunization of mice with ovalbumin containing immune complexes induces proliferative expansion of ovalbumin specific T cells, which however fail to inhibit tumor growth and lack effector function when restimulated in vitro. B16 is the murine melanoma model that does not express ovalbumin (OVA). B16 melanoma-bearing mice will be treated with the mAb TA99+/−ONTAK.

ONTAK pre-treated and untreated WT mice will be immunized with ovalbumin containing immune complexes. Two weeks later mice will be challenged with B16-OVA and monitored for tumor growth.

In the HER-2/neu Transgenic (Tg) mouse model, mice spontaneously develop breast cancers by 6 months of age. Weekly treatment with anti-HER-2 mAbs delays tumor development in these mice until 9 months of age. Therapy with ONTAK will be combined to extend the treatment effect. Three experimental protocols are as follows:

a) Mice will be administered Anti-HER2 antibodies with or without ONTAK, representing a chronic treatment model;

b) Mice will be administered Anti-HER2 immune complexes vaccine with or without ONTAK representing a spontaneous tumor development model; and

c) Mice will be administered Anti-HER2 immune complexes vaccine with or without ONTAK in WT mice challenged with HER-2 expressing tumors.

Endpoints to be evaluated include: tumor protection, OVA and HER-2 specific T cell responses.

What is claimed:

1. A method for converting a passive immunization against a target antigen into active immunity against the target antigen in a subject, the method comprising:
(a) administering an effective amount of an agent, wherein the agent decreases the activity or function of a regulatory T cell or substantially depletes the regulatory T cell population in the subject, and
(b) increasing immune complex formation or immune complex number in the subject, wherein the immune complex comprises (i) an antibody or antibody fragment that comprises at least a portion of an immunoglobulin variable region that specifically binds to the target antigen and at least a portion of immunoglobulin constant region that can bind to an Fc-receptor;
thereby inducing, activating, or stimulating T helper and or T cytotoxic cells that have T cell receptors specific to the target antigen in the subject.

2. The method of claim 1, wherein the step of increasing immune complex formation or immune complex number in the subject comprises administering to the subject: (a) the antibody or antibody fragment such that the antibody or antibody fragment forms immune complexes with its target antigen in the subject, and/or (b) immune complexes that comprise the antibody or antibody fragment and the target antigen.

3. The method of claim 2, wherein the antibody, antibody fragment, and/or immune complexes are co-administered with the agent in an amount effective to induce, activate, or stimulate T helper and or T cytotoxic cells that have T cell receptors specific to the target antigen in the subject.

4. The method of claim 1, wherein the subject has cancer or a pathogenic infection.

5. The method of claim 4, wherein the cancer is selected from B cell lymphoma, colon cancer, lung cancer, renal cancer, bladder cancer, T cell lymphoma, myeloma, leukemia, chronic myeloid leukemia, acute myeloid leukemia, chronic lymphocytic leukemia, acute lymphocytic leukemia, hematopoietic neoplasias, thymoma, lymphoma, sarcoma, lung cancer, liver cancer, non-Hodgkins lymphoma, Hodgkins lymphoma, uterine cancer, renal cell carcinoma, hepatoma, adenocarcinoma, breast cancer, pancreatic cancer, liver cancer, prostate cancer, head and neck carcinoma, thyroid carcinoma, soft tissue sarcoma, ovarian cancer, primary or metastatic melanoma, squamous cell carcinoma, basal cell carcinoma, brain cancer, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, astrocytoma, medulloblastoma, craniopharyngioma, ependymoma, pinealoma, hemangioblastoma, acoustic neuroma, oligodendroglioma, meningioma, retinoblastoma, leukemia, melanoma, neuroblastoma, small cell lung carcinoma, bladder carcinoma, lymphoma, multiple myeloma, and medullary carcinoma.

6. The method of claim 4, wherein the pathogenic infection is caused by a bacterium, parasite, virus, fungus, or protozoa.

7. The method of claim 1, wherein the subject is a mammal.

8. The method of claim 7, wherein the subject is a human.

9. The method of claim 1, wherein the agent is ONTAK, HuMax-Tac, Zenapax, or MDX-010 or a combination thereof.

10. The method of claim 1, wherein the agent is an antibody or a fragment thereof which specifically binds to a T regulatory cell surface protein.

11. The method of claim 10, wherein the T regulatory cell surface protein is CD25 or CTLA4.

12. The method of claim 10, wherein the antibody or fragment thereof further comprises a radionuclide or toxic moiety.

13. The method of claim 10, wherein the surface protein comprises CD25, CD4, CD28, CD38, CD62L (selectin), OX-40 ligand (OX-40L), CTLA4, CCR4, CCR8, FOXP3, LAG3, CD103, NRP-1, or glucocorticoid-induced TNF receptor (GITR).

14. The method of claim 1, wherein the agent is a fusion protein.

15. The method of claim 14, wherein the fusion protein comprises a targeting moiety and a toxic moiety.

16. The method of claims 15, wherein the targeting moiety is a ligand of a regulatory T cell surface protein.

17. The method of claim 16, wherein the ligand is IL2, T cell receptor (TCR), MHCII, CD80, CD86, TARC, CCL17, CKLF1, CCL1, TCA-3, eotaxin, TER-1, E-cadherin, VEGF, semaphorin3a, CD134, CD31, CD62, CD38L, or glucocorticoid-induced TNF receptor ligand (GITRL).

18. The method of claim 12 or 15, wherein the toxic moiety comprises lectin, ricin, abrin, viscumin, modecin, diphtheria toxin, cholera toxin, gelonin, Pseudomonas exotoxin, Shigella toxin, botulinum toxin, tetanus toxin, calicheamicin, or pokeweed antiviral protein.

19. The method of claim 1, wherein the target antigen is a cancer antigen.

20. The method of claim 19, wherein the cancer antigen is selected from: HER2, BRCA1, prostate-specific membrane antigen (PSMA), MART-1/MelanA, prostatic serum antigen (PSA), squamous cell carcinoma antigen (SCCA), ovarian cancer antigen (OCA), pancreas cancer associated antigen (PaA), MUC-1, MUC-2, MUC-3, MUC-18, carcino-embryonic antigen (CEA), polymorphic epithelial muc in (PEM), Thomsen-Friedenreich (T) antigen, gp100, tyrosinase, TRP-1, TRP-2, NY-ESO-1, CDK-4, β-catenin, MUM-1, Caspase-8, KIAA0205, HPVE7, SART-1, SART-2, PRAME, BAGE-1, DAGE-1, RAGE-1, NAG, TAG-72, CA125, mutated p21ras, mutated p53, HPV16 E7, RCC-3.1.3, MAGE-1, MAGE-2, MAGE-3, MAGE-4, MAGE-11, GAGE-I, GAGE-6, GD2, GD3, GM2, TF, sTn, gp75, EBV-LMP 1, EBV-LMP 2, HPV-F4, HPV-F6, HPV-F7, alpha-fetoprotein (AFP), CO17-1A, GA733, gp72, p-HCG, gp43, HSP-70, p17 mel, HSP-70, gp43, HMW, HOJ-1, HOM-MEL-55, NY-COL-2, HOM-HD-397, HOM-RCC-1.14, HOM-HD-21, HOM-NSCLC-11, HOM-MEL-2.4, HOM-TES-11, melanoma gangliosides, TAG-72, prostatic acid phosphatase, protein MZ2-E, folate-binding-protein LK26, truncated epidermal growth factor receptor (EGFR), GM-2 and GD-2 gangliosides, polymorphic epithelial mucin, folate-binding protein LK26, pancreatic oncofetal antigen, cancer antigen 15-3, cancer antigen 19-9, cancer antigen 549, or cancer antigen 195.

21. The method of claim 1, wherein the target antigen is an antigen from a pathogen.

22. The method of claim 21, wherein the antigen is a viral antigenic peptide or protein.

23. The method of claim 22, wherein the viral antigenic peptide or protein is expressed by Arboviruses, Herpesviruses, herpes simplex viruses, Epstein Barr virus, cytomegalovirus, varicella-zoster virus, human herpes virus 6, human herpes virus 8, herpes B virus Hepadnaviruses, hepatitis virus A, B, C, D, E, F, or G, Togaviruses, Venezuelan equine encephalitis virus, Coronaviruses, severe acute respiratory syndrome virus, Picornaviruses, polioviruses, Flaviviruses, human hepatitis C virus, yellow fever virus, dengue viruses, Retroviruses, human immunodeficiency viruses, human T lymphotropic viruses, Paramyxoviruses, respiratory syncytial virus, Reoviruses, rotaviruses, Bunyaviruses, hantaviruses, Filoviruses, Ebola virus, Adenoviruses, Parvoviruses, parvovirus B-19; Papovaviruses, human papilloma viruses, Rhabdoviruses, rabies virus, Arenaviruses, Lassa virus, Orthomyxoviruses, influenza viruses, Poxviruses, Orf virus, molluscum contageosum virus, Canine distemper virus, Canine contagious hepatitis virus, Feline calicivirus, Feline rhinotracheitis virus, TGE virus, smallpox virus, Monkey pox virus, rhinoviruses, orbiviruses, picodnaviruses, encephalomyocarditis virus, Parainfluenza viruses, adenoviruses, Coxsackieviruses, Echoviruses, Rubeola virus, Rubella virus, human metapneuomovirus, enteroviruses, Foot and mouth disease virus, simian virus 5, or human parainfluenza virus type 2.

24. The method of claim 21, wherein the antigen is a bacterial antigenic peptide or protein.

25. The method of claim 24, wherein the bacterial antigenic peptide or protein is expressed by Mycoplasma sp., Ureaplasma sp., Neisseria sp., Treponema sp., Bacillus sp., Haemophilus sp., Rickettsia sp., Chlamydia sp., Corynebacterium sp., Mycobacterium sp., Clostridium sp., Legionella sp., Shigella sp., Salmonella sp., pathogenic Escherichia sp., Vibrio sp., Staphylococcus sp., Bordatella sp., Moraxella sp., Streptococcus sp., Campylobacter sp., Borrelia sp., Leptospira sp., Pseudomonas sp., Helicobacter sp., Erlichia sp., or Klebsiella sp.

26. The method of claim 21, wherein the antigen is a fungal antigenic peptide or protein.

27. The method of claim 26, wherein the fungal antigenic peptide or protein is expressed by Aspergillus sp., Pneumocystis sp. (such as P. carinii), Tinea sp., Candida sp., Sporothrix sp., Cryptococcus sp., Histoplasma sp., or Coccidioides sp.

28. The method of claim 21, wherein the antigen is a protozoan or parasitic antigenic peptide or protein.

29. The method of claim 28, wherein the protozoan antigenic peptide or protein is expressed by Trypanosoma sp., Endamoeba sp., Giardia sp., Plasmodium sp., Babeosis sp., Toxoplasma sp., or Leishmania sp.

30. The method of claim 28, wherein the parasitic antigenic peptide or protein is expressed by Schistosoma sp., Taenia sp., Echinococcus sp., Hymenolepsis sp., Diphyllobotrium sp., Fasciolopsis sp., Trichinella sp., or Ascaris sp.

31. The method of claim 1, wherein steps (a) and (b) are conducted simultaneously.

32. The method of claim 1, wherein steps (a) and (b) are conducted sequentially in any order.

33. The method of claim 1, further comprising administering a vaccine that comprises the target antigen.

34. The method of claim 1 further comprising administering a chemotherapy drug, an antibiotic, an antifungal drug, an antiviral drug, anti-parasitic drug, or an anti-protozoal drug or a combination thereof.

35. The method of claim 34, wherein the chemotherapy drug is an alkylating agent, a nitrosourea, an anti-metabolite, a topoisomerase inhibitor, a mitotic inhibitor, an anthracycline, a corticosteroid hormone, a sex hormone, or a targeted anti-tumor compound or a combination thereof.

36. The method of claim 34, wherein the targeted anti-tumor compound is imatinib (Gleevec), gefitinib (Iressa), erlotinib (Tarceva), rituximab (Rituxan), or bevacizumab (Avastin).

37. The method of claim 35, wherein the alkylating agent is busulfan, cisplatin, chlorambucil, cyclophosphamide (Cytoxan), dacarbazine (DTIC), mechlorethamine, melphalan, or temozolomide.

38. The method of claim 35, wherein the anti-metabolite is 5-fluorouracil or methotrexate.

39. The method of claim 35, wherein the topoisomerase inhibitor is topotecan, etoposide, or teniposide.

40. The method of claim 35, wherein the anthracycline is daunorubicin, doxorubicin, epirubicin, idarubicin, or mitoxantrone.

41. A method for treating or reducing cancer in a subject, the method comprising:
(a) administering an effective amount of an agent, wherein the agent decreases the activity or function of a regulatory T cell or substantially depletes the regulatory T cell population in the subject, and
(b) increasing immune complex formation or immune complex number in the subject, wherein the immune complex comprises (i) an antibody or antibody fragment that comprises at least a portion of an immunoglobulin variable region that specifically binds to a tumor antigen and at least a portion of immunoglobulin constant region that can bind to an Fc-receptor;
thereby inducing, activating, or stimulating T helper and or T cytotoxic cells that have T cell receptors specific to the tumor antigen in the subject.

42. The method of claim 41, further comprising administering a chemotherapy drug.

43. The method of claim 41, wherein the cancer is selected from B cell lymphoma, colon cancer, lung cancer, renal cancer, bladder cancer, T cell lymphoma, myeloma, leukemia, chronic myeloid leukemia, acute myeloid leukemia, chronic lymphocytic leukemia, acute lymphocytic leukemia, hematopoietic neoplasias, thymoma, lymphoma, sarcoma, lung cancer, liver cancer, non-Hodgkins lymphoma, Hodgkins lymphoma, uterine cancer, renal cell carcinoma, hepatoma, adenocarcinoma, breast cancer, pancreatic cancer, liver cancer, prostate cancer, head and neck carcinoma, thyroid carcinoma, soft tissue sarcoma, ovarian cancer, primary or metastatic melanoma, squamous cell carcinoma, basal cell carcinoma, brain cancer, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, astrocytoma, medulloblastoma, craniopharyngioma, ependymoma, pinealoma, hemangioblastoma, acoustic neuroma, oligodendroglioma, meningioma, retinoblastoma, leukemia, melanoma, neuroblastoma, small cell lung carcinoma, bladder carcinoma, lymphoma, multiple myeloma, and medullary carcinoma.

44. The method of claim 41, wherein the agent is ONTAK, HuMax-Tac, Zenapax, or MDX-010 or a combination thereof.

45. The method of claim 41, wherein the agent comprises an antibody or a fragment thereof directed at a cell surface protein of a regulatory T cell.

46. The method of claim 45, wherein the antibody or fragment thereof further comprises a radionuclide or toxic moiety.

47. The method of claim 46, wherein the radionuclide is iodine-131, yttrium-90, rhodium-186, astatine-211, or bismuth-213.

48. The method of claim 45, wherein the antibody or a fragment thereof binds to CD25, CD4, CD28, CD38, CD62L (selectin), OX-40 ligand (OX-40L), CTLA4, CCR4, CCR8, FOXP3, LAG3, CD103, NRP-1, or glucocorticoid-induced TNF receptor (GITR).

49. The method of claim 45, wherein the agent is a fusion protein.

50. The method of claim 49, wherein the fusion protein comprises a targeting moiety and a toxic moiety.

51. The method of claim 50, wherein the targeting moiety is a ligand of a regulatory T cell surface protein.

52. The method of claim 51, wherein the ligand is IL2, T cell receptor (TCR), MHCII, CD80, CD86, TARC, CCL17, CKLF1, CCL1, TCA-3, eotaxin, TER-1, E-cadherin, VEGF, semaphorin3a, CD134, CD31, CD62, CD38L, or glucocorticoid-induced TNF receptor ligand (GITRL).

53. The method of claim 46 or 50, wherein the toxic moiety comprises lectin, ricin, abrin, viscumin, modecin, diphtheria toxin, cholera toxin, gelonin, Pseudomonas exotoxin, Shigella toxin, botulinum toxin, tetanus toxin, calicheamicin, or pokeweed antiviral protein.

54. The method of claim 51, wherein the step of increasing immune complex formation or immune complex number in the subject comprises administering to the subject: (a) the antibody or antibody fragment such that the antibody or antibody fragment forms immune complexes with the tumor antigen in the subject, and/or (b) immune complexes that comprise the antibody or antibody fragment and the tumor antigen.

55. The method of claim 54, wherein the antibody, antibody fragment, and/or immune complexes are co-administered with the agent in an amount effective to induce, activate, or stimulate T helper and or T cytotoxic cells that have T cell receptors specific to the tumor antigen in the subject.

56. The method of claim 41, wherein the tumor antigen is selected from: HER2, BRCA1, prostate-specific membrane antigen (PSMA), MART-1/MelanA, prostatic serum antigen (PSA), squamous cell carcinoma antigen (SCCA), ovarian cancer antigen (OCA), pancreas cancer associated antigen (PaA), MUC-1, MUC-2, MUC-3, MUC-18, carcino-embryonic antigen (CEA), polymorphic epithelial muc in (PEM), Thomsen-Friedenreich (T) antigen, gp100, tyrosinase, TRP-1, TRP-2, NY-ESO-1, CDK-4, β-catenin, MUM-1, Caspase-8, KIAA0205, HPVE7, SART-1, SART-2, PRAME, BAGE-1, DAGE-1, RAGE-1, NAG, TAG-72, CA125, mutated p21ras, mutated p53, HPV16 E7, RCC-3.1.3, MAGE-1, MAGE-2, MAGE-3, MAGE-4, MAGE-11, GAGE-I, GAGE-6, GD2, GD3, GM2, TF, sTn, gp75, EBV-LMP 1, EBV-LMP 2, HPV-F4, HPV-F6, HPV-F7, alpha-fetoprotein (AFP), CO17-1A, GA733, gp72, p-HCG, gp43, HSP-70, p17 mel, HSP-70, gp43, HMW, HOJ-1, HOM-MEL-55, NY-COL-2, HOM-HD-397, HOM-RCC-1.14, HOM-HD-21, HOM-NSCLC-11, HOM-MEL-2.4, HOM-TES-11, melanoma gangliosides, TAG-72, prostatic acid phosphatase, protein MZ2-E, folate-binding-protein LK26, truncated epidermal growth factor receptor (EGFR), GM-2 and GD-2 gangliosides, polymorphic epithelial mucin, folate-binding protein LK26, pancreatic oncofetal antigen, cancer antigen 15-3, cancer antigen 19-9, cancer antigen 549, or cancer antigen 195.
